(19) United States
(12) Patent Application Publication
(10) Pub. No.: US 2003/0125241 A1
(43) Pub. Date: Jul. 3, 2003

(54) Therapeutic uses of LNA-modified oligonucleotides in infectious diseases

(76) Inventors: Margit Wissenbach, Fredensborg (DK); Troels Koch, Copenhagen (DK); Henrik Orum, Vaerlose (DK); Bo Hansen, Copenhagen (DK)

Correspondence Address: EDWARDS & ANGELL, LLP, P.O. BOX 9169, BOSTON, MA, 02209, US

(21) Appl. No.: 10/150,779

(22) Filed: May 17, 2002

Related U.S. Application Data
(60) Provisional application No. 60/291,830, filed on May 18, 2001.

(51) Int. Cl. A61K 48/00

(52) U.S. Cl. 514/8; 514/44

(57) ABSTRACT

The invention relates to therapeutic applications of LNA-modified oligonucleotides. In particular, the invention provides methods for treatment of infectious diseases and disorders caused by viruses, bacteria, protozoa or fungi. Preferably, administration of an LNA-modified oligonucleotide modulates expression of a targeted gene associated with the replication or infectivity of a virus, virulence genes, host immune modulating genes and the like. That is, preferred use of LNA-modified oligonucleotide provides an antisense-type therapy with selective modulation of gene expression of predetermined targets.

This application claims the benefit of U.S. Provisional application No. 60/291,830 filed May 18, 2001, the entirety of which is incorporated by reference herein.

### BACKGROUND OF THE INVENTION

1. Field of the Invention

The invention relates to therapeutic applications of LNA-modified oligonucleotides. In particular, the invention provides methods for treatment of infectious diseases and disorders caused or associated with by viruses, bacteria, protozoa or fungi. Preferably, administration of an LNA-modified oligonucleotide modulates expression of a targeted gene that is related to the replication, infectivity or survival of the organism in question, a virulence gene or host immune modulating genes and the like. Preferred use of LNA-modified oligonucleotide provides an antisense-type therapy with selective modulation of gene expression of predetermined targets.

2. Background

Certain nucleotide-based compounds have been utilized in various therapeutic applications. In particular, various oligonucleotides have been investigated including single stranded and double stranded oligonucleotides, and analogues. To be useful in in vivo applications an oligonucleotide must have a plethora of properties including the ability to penetrate a cell membrane, have good resistance to extra- and intracellular nucleases, have high affinity and specificity for the target and preferably have the ability to recruit endogenous enzymes such as RNAseH, RNAaseIII, RNAseL etc.

A fundamental property of oligonucleotides that underlies many of their potential therapeutic applications is their ability to recognize and hybridize specifically to complementary single stranded nucleic acids employing either Watson-Crick hydrogen bonding (A—T and G—C) or other hydrogen bonding schemes such as the Hoogsteen/reverse Hoogsteen mode. Affinity and specificity are properties commonly employed to characterize hybridization characteristics of a particular oligonucleotide. Affinity is a measure of the binding strength of the oligonucleotide to its complementary target (expressed as the thermostability (Tₘ) of the duplex). Each nucleobase pair in the duplex adds to the thermostability and thus affinity increases with increasing size (No. of nucleobases) of the oligonucleotide. Specificity is a measure of the ability of the oligonucleotide to discriminate between a fully complementary and a mismatched target sequence. In other words, specificity is a measure of the loss of affinity associated with mismatched nucleobase pairs in the target.

Certain conformational restriction has been applied in recent years to oligonucleotides in the search for analogues displaying improved hybridization properties compared to unmodified (2′-deoxy) oligonucleotides. For instance, there have been reported bicyclo[3.3.0]nucleosides with an additional C-3′,C-5′-ethano-bridge (see e.g., M. Tarköy et al., Helv. Chim. Acta, 1993, 76, 481); bicarbocyclo[3.1.0]nucleosides with an additional C-1′,C-6′- or C-6′,C-4′methano bridge (see e.g., K.-H. Altmann et al., Tetrahedron Lett., 1994, 35, 2331); bicyclo[3.3.0]- and [4.3.0]nucleosides containing an additional C-2′,C-3′-dioxalane ring synthesized as a dimer with an unmodified nucleoside where the additional ring is part of the internucleoside linkage replacing a natural phosphodiester linkage (see e.g., R. J. Jones et al., J. Am. Chem. Soc., 1993, 115, 9816); dimers containing a bicyclo[3.1.0]nucleoside with a C-2′,C-3′-methano bridge as part of amide- and sulfonamide-type internucleoside linkages (see e.g., C. G. Yannopoulus et al., Synlett, 1997, 378); bicyclo[3.3.0]glucose-derived nucleoside analogue incorporated in the middle of a trimer through formacetal internucleoside linkages (see e.g., C. G. Yannopoulus et al., Synlett, 1997, 378; tricyclo-DNA in which two five membered rings and one three membered ring constitute the backbone (see R. Steffens & C. J. Leumann, J. Am. Chem. Soc, 1997, 119, 11548-49); 1,5-Anhydrohexitol nucleic acids (see Aerschot et al., Angew. Chem. Int. Ed. Engl. 1995, 34(129 1338-39); and bicyclic[4.3.0]- and [3.3.0]nucleosides with additional C-2′,C-3′-connected six and five-membered ring; (see e.g., P. Nielsen et al., XII International Roundtable: Nucleosides, Nucleotides and Their Biological Applications, La Jolla, Calif., Sep. 15-19, 1996, Poster PPI 43). However, oligonucleotides comprising these analogues form, in most cases, less stable duplexes with complementary nucleic acids compared to the unmodified oligonucleotides.

Recently, novel DNA compounds referred to as Locked Nucleic Acids (LNA) have been reported (see International Patent Application WO 99/14226; P. Nielsen et al, J. Chem. Soc., Perkin Trans. 1, 1997, 3423; P. Nielsen et al., Chem. Commun., 1997, 9, 825; N. K. Christensen et al., J. Am. Chem. Soc., 1998, 120, 5458; A. A. Koshkin et al., J. Org. Chem., 1998, 63, 2778; A. A Koshkin et al. J. Am. Chem. Soc. 1998, 120, 13252-53; Kumar et al. Bioorg, & Med. Chem. Lett., 1998, 8, 2219-2222; and S. Obika et al., Bioorg. Med. Chem. Lett., 1999, 515). Interestingly, incorporation of LNA monomers containing a 2′-O,4′-C-methylene bridge into an oligonucleotide sequence led to an unprecedented improvement in the hybridization stability of the modified oligonucleotide (see above and e.g., S. K. Singh et al., Chem. Commun., 1998, 455). Oligonucleotides comprising the 2′-O,4′-C-methylene bridge (LNA) monomers and also the corresponding 2′-thio-LNA (thio-LNA), 2′-HN-LNA (amino-LNA), and 2′-N(R)-LNA (amino-R-LNA) analogue, form duplexes with complementary DNA and RNA with thermal stabilities not previously observed for bi- and tricyclic nucleosides modified oligonucleotides. The increase in Tₘ per modification varies from +3 to +11° C., and furthermore, the selectivity is also improved. No other DNA analogue has reproducibly shown such high affinity for nucleic acids.

Molecular strategies are being developed to modulate unwanted gene expression that either directly causes, participates in, or aggravates a disease state. One such strategy involves inhibiting gene expression with oligonucleotides complementary in sequence to the messenger RNA of a deleterious target gene. The messenger RNA strand is a copy of the coding DNA strand and is therefore, as the DNA strand, called the sense strand. Oligonucleotides that hybridize to the sense strand are called antisense oligonucleotides. Binding of these strands to mRNA interferes with the translation process and consequently with gene expression.

Antisense strategies have been used in infections caused by viruses such as inhibition of HIV replication. Zamecnic and coworkers have used phosphodiester oligonucleotides targeted to the reverse transcriptase primer site and to splice donor/acceptor sites; P. C. Zamecnik, J. Goodchild, Y. Taguchi, P. S. Sarin, Proc. Natl. Acad. Sci. USA 83, 4143 (1986). Goodchild and coworkers have made phosphodiester compounds targeted to the initiation sites for translation, the cap site, the polyadenylation signal, the 5′ repeat region and a site between the gag and pol genes; J. Goodchild, S. Agrawal, M. P. Civeira, P. S. Sarin, D. Sun, P. C. Zamecnik, Proc. Natl. Acad. Sci. U.S.A. 85, 5507 (1988). Antisense oligonucleotides have also been used against other viral agents and also bacterial agents by targeting, and down regulating the replication of Hepatitis B virus (see e.g. U.S. Pat. No. 5,985,662), Hepatitis C (see e.g. WO9703211), Herpes viruses (see e.g. U.S. Pat. No. 5,658,891 and U.S. Pat. No. 5,248,670), Influenza viruses (see e.g. U.S. Pat. No. 5580767) and H. pylori (see e.g. U.S. Pat. No. 6,124,271).

These prior attempts at targeting infectious agents have largely focused on the nature of the chemical modification used in the oligonucleotide analog. Although most of the above publications have reported some degree of success in inhibiting some function of the virus, a general therapeutic scheme to target infectious agents has not been found. Accordingly, there has been and continues to be a long-felt need for the design of oligonucleotides and oligonucleotide analogs which are capable of effective, therapeutic antisense use.

### SUMMARY OF THE INVENTION

The present invention provides use of LNA-modified oligonucleotides for treatment of infectious diseases such as for example those associated with viral, bacterial, protozoan or fungal infections.

Preferably, an LNA-modified oligonucleotide (or simply LNA oligonucleotide) is employed that enables effective modulation of the expression (translation) of a specific gene(s). As such the invention provides means to develop drugs against diseases in which a normal gene product is involved in a pathophysiological process or diseases that stem from the presence of infectious agents.

The invention may be used against protein coding genes as well as non-protein coding genes. Examples of non-protein coding genes include genes that encode ribosomal RNAs, transfer RNAs, small nuclear RNAs, small cytoplasmic RNAs, telomerase RNA, RNA molecules involved in DNA replication, chromosomal rearrangement of, for instance immunoglobulin genes, etc.

According to one preferred embodiment of the invention, the LNA-modified antisense oligonucleotide is specific for genes responsible for viral replication; a viral infection cycle, such as, for example, attachment to cellular ligands; viral genes encoding host immune modulating functions. Particularly preferred viral organisms causing human diseases according to the present invention include (but not restricted to) Herpes viruses, Hepatitisviruses, Retroviruses, Orthomyxoviruses, Paramyxoviruses, Togaviruses, Picomaviruses, Papovaviruses and Gastroenteritisviruses.

According to another preferred embodiment of the invention, the LNA-modified antisense oligonucleotide is specific for human or domestic animal bacterial pathogens. Particularly preferred bacteria causing serious human diseases are the Gram positive organisms: Staphylococcus aureus, Staphylococcus epidermidis, Enterococcus faecalis and E. faecium, Streptococcus pneumoniae and the Gram negative organisms: Pseudomonas aeruginosa, Burkholdia cepacia, Xanthomonas maltophila, Escherichia coli, Enterobacter spp, Klebsiella pneumoniae and Salmonella spp. The target genes may include (but are not restricted to) genes essential to bacterial survival and multiplication in the host organism, virulence genes, genes encoding single- or multi-drug resistance.

According to one preferred embodiment of the invention, the LNA-modified antisense oligonucleotide is specific for protozoa infecting humans and causing human diseases. Particularly preferred protozoan organisms causing human diseases according to the present invention include (but not restricted to) Malaria e.g. Plasmodium falciparum and M. ovale, Trypanosomiasis (sleeping sickness) e.g. Trypanosoma cruzei, Leischmaniasis e.g. Leischmania donovani, Amebiasis e.g. Entamoeba histolytica.

According to one preferred embodiment of the invention, the LNA-modified antisense oligonucleotide is specific for fungi causing pathogenic infections in humans. Particularly preferred fungi causing or associated with human diseases according to the present invention include (but not restricted to) Candida albicans, Histoplasma neoformans, Coccidioides immitis and Penicillium marneffei.

The invention in general provides a method for treating diseases which are caused by infectious agents such as viruses, bacteria, intra- and extra-cellular parasites, insertion elements, fungal infections, etc., which may also cause expression of genes by a normally unexpressed gene, abnormal expression of a normally expressed gene or expression of an abnormal gene, comprising administering to a patient in need of such treatment an effective amount of an LNA-modified antisense oligonucleotide; or a cocktail of different LNA-modified antisense oligonucleotides; or a cocktail of different LNA-modified and unmodified antisense oligonucleotides specific for the disease causing entity.

An LNA-modified oligonucleotide contains one or more units of an LNA monomer, preferably one or more 2′-O,4′-C-methylene bridge monomers (oxy-LNA). An LNA-modified oligonucleotide however also may contain other LNA units in addition to or in place of an oxy-LNA group. In particular, preferred additional LNA units include 2′-thio-LNA (thio-LNA), 2′-HN-LNA (amino-LNA), and 2′-N(R)-LNA (amino-R-LNA)) monomers in either the D-β or L-α configurations or combinations thereof. An LNA-modified oligonucleotide also may have other internucleoside linkages than the native phosphodiester, e.g. phosphoromonothioate, phosphorodithioate, and methylphosphonate linkages. The LNA-modified oligonucleotide can be fully modified with LNA (i.e. each nucleotide is an LNA unit), but it is generally preferred that the LNA-modified oligomers contain other residues such as native DNA monomers, phosphoromonothioate monomers, methylphosphonate monomers or analogs thereof. In general, an LNA-modified oligonucleotide will contain at least about 5, 10, 15 or 20 percent LNA units, based on total nucleotides of the oligonucleotide, more typically at least about 20, 25, 30, 40, 50, 60, 70, 80 or 90 percent LNA units, based on total bases of the oligonucleotide. However, the LNA-modified oligonucleotide may also be fully modified as showed in Example 3.

An LNA-modified oligonucleotide used in accordance with the invention suitably is at least a 5-mer, 6-mer, 7-mer, 8-mer, 9-mer or 10-mer oligonucleotide, that is, the oligonucleotide is an oligomer containing at least 5, 6, 7, 8, 9, or 10 nucleotide residues, more preferably at least about 11 or 12 nucleotides. The preferred maximum size of the oligonucleotide is about 40, 50 or 60 nucleotides, more preferably up to about 25 or 30 nucleotides, and most preferably from about between 12 and 20 nucleotides. While oligonucleotides smaller than 10-mers or 12-mers may be utilized they are more likely to hybridize with non-targeted sequences (due to the statistical possibility of finding exact sequence matches by chance in the human genome), and for this reason may be less specific. In addition, a single mismatch may destabilize the hybrid thereby impairing its therapeutic function. While oligonucleotides larger than 40-mers may be utilized, synthesis, and cellular uptake may become somewhat more troublesome. Specialized vehicles or oligonucleotide carriers are known in the art for improving cellular uptake of large oligomers. Moreover, partial matching of long sequences may lead to non-specific hybridization, and non-specific effects.

In principle, oligonucleotides having a sequence complementary to any region of the target mRNA have utility in the present invention. In one embodiment of the invention oligonucleotides are capable of forming a stable duplex with a portion of the transcript lying within about 50 nucleotides (preferably within about 40 nucleotides) upstream (the 5′ direction), or about 50 (preferably 40) nucleotides downstream (the 3′ direction) from the translation initiation codon of the target mRNA. In another embodiment, preferred oligonucleotides include those oligonucleotides which are capable of forming a stable duplex with a portion of the target mRNA transcript including the translation initiation codon.

LNA-modified oligonucleotides are useful for a number of therapeutic applications as indicated above. In general, therapeutic methods of the invention include administration of a therapeutically effective amount of an LNA-modified oligonucleotide to a mammal, particularly a human.

In antisense therapies, administered LNA-modified oligonucleotide contacts (interacts with) the targeted gene or mRNA from the gene, whereby expression of the gene is modulated, and frequently expression is inhibited rather than increased. Such modulation of expression suitably can be a difference of at least about 10% or 20% relative to a control, more preferably at least about 30%, 40%, 50%, 60%, 70%, 80%, or 90% difference in expression relative to a control. It will be particularly preferred where interaction or contact with an LNA-modified oligonucleotide results in complete or essentially complete modulation of expression relative to a control, e.g., at least about a 95%, 97%, 98%, 99% or 100% inhibition of or increase in expression relative to control. A control sample for determination of such modulation can be comparable cells (in vitro or in vivo) that have not been contacted with the LNA-modified oligonucleotide.

The methods of the invention are preferably employed for treatment or prophylaxis against diseases caused by infectious agents, particularly for treatment of infections as may occur in tissue such as lung, heart, liver, prostate, brain, testes, stomach, intestine, bowel, spinal cord, sinuses, urinary tract or ovaries of a subject. The methods of the invention also may be employed to treat systemic conditions such as viremia or septicemia. The methods of the invention are also preferably employed for treatment of diseases and disorders associated with viral infections or bacterial infections, as well as any other disorder caused by an infectious agent.

In another aspect, the invention provides use of the disclosed LNA oligonucoetides for the preparation of a medicament net useful for the treatment of a viral, bacterial, protozoa or fungal infection, or a disease or disorder associated therewith.

The invention also provides pharmaceutical composition that comprise an LNA oligonucleotide as disclosed herein, preferably packaged with written instructions for use of the oligonucleotide, particularly to treat against a viral, bacterial, protozoa or fungal infection, or a disease or disorder associated therewith.

Other aspects of the invention are disclosed infra.

### BRIEF DESCRIPTION OF THE DRAWINGS

FIG. 1 is a bar graph which shows the effect of the fully modified LNA oligonucleotide on the steady state expression of the human GAPDH gene, using lipofectin (12 μg/ml ) as a transfection vehicle.

FIG. 2 is a bar graph which shows the effect of the fully modified LNA oligonucleotide on the steady state expression of the human GAPDH gene in the absence of a transfection vehicle.

FIG. 3 is a schematic illustration of the human mRNA coding for the High affinity IgE receptor Fc epsilon RI alpha-chain (FcεRIα) was used as a model for in vitro replicational arrest. Fully modified oxy-LNA 16 mer oligonucleotides complementary to the 3′-region (Cur 0089, Cur 0106, Cur 0112) or the 5′ region of the cDNA were used.

FIG. 4 is a graph which shows the dose response effect of the fully oxy-LNA modified oligonucleotides on full length reverse transcription. Data are obtained by real time PCR using a Taqman assay which lies upstream of Cur 0089, Cur 0106, Cur 0112 and downstream of Cur 0087.

FIG. 5 is a graph showing the translational inhibition of the antisense anti-HCV LNA modified oligonucleotide, compared to the control.

### DETAILED DESCRIPTION OF THE INVENTION

In a first aspect, the invention provides methods for treating cells comprising an infectious agent such as those discussed above. Such treatment methods comprise administering an LNA-modified oligonucleotide to cells that comprise an oligonucleotide sequence of an infectious agent. The LNA-modified oligonucleotide preferably will be complementary to the infectious agent oligonucleotide sequence. A variety of cells may be treated in accordance with such methods, and typically mammalian cells are treated, especially primate cells such as human cells.

Preferred LNA oligonucleotides of the invention will hybridize (bind) to a target sequence, particularly a target oligonucleotide of an infectious agent such as a viral, bacterial, fungal or protozoan agent including those agents and sequences disclosed herein, under high stringency conditions as may be assessed in vitro. Such conditions are disclosed and defined below.

According to the preferred present invention, an LNA modified oligonucleotide is designed to be specific for a gene, which either causes, participates in, or aggravates a disease state. This can be achieved by i) reducing or inhibiting the expression of the involved gene(s) or by ii) inducing or increasing the expression of a normally lowly expressed or unexpressed gene(s) the expression of which may mitigate or cure the disease state. Such induction or increase in the expression of a target gene may be achieved by, for instance, directing an antisense oligonucleotide against the mRNA of a gene that encodes a natural repressor of the target gene, by designing the antisense oligonucleotide in such a way that binding to its complementary sequence in the target mRNA will lead to an increase in target mRNA half-life and expression, or by using an antigene oligonucleotide that can strand invade double stranded DNA to form a complex that can function as an initiation point for transcription of a downstream gene as described in Møllegaard et al. Proc. Natl. Acad. Sci. USA, 1994, 91(9), 3892-3895.

As used herein, “contact” refers to the high affinity binding of LNA-modified oligonucleotides to infectious disease causing agents' target nucleic acid sequences. The high affinity binding, as measured by Tₘ and hybridization stringency, has an association constant (Kₐ) between the LNA-modified oligonucleotide and target sequence that is higher than the association constant of a complementary strand of the infectious agent target nucleic acid sequence, and this association constant is higher than the disassociation constant (K_{d}) of a complementary strand of the target nucleic acid sequence.

Antisense compounds, in accordance with the invention include, but are not limited to ribozymes, aptamers, siRNA, external guide sequence (EGS) oligonucleotides (oligozymes), and other short catalytic RNAs or catalytic oligonucleotides which hybridise to the target nucleic acid and modulate its expression. Aptamers are a promising new class of therapeutic oligonucleotides and are selected in vitro to specifically bind to a given target with high affinity, such as for example ligand receptors. Their binding characteristics are likely a reflection of the ability of oligonucleotides to form three dimensional structures held together by intramolecular nucleobase pairing. Ribozymes are RNA molecules that have a catalytic activity, and can be comprised of oligoribonucleotides and oligodeoxyribonucleotides and analogues thereof. Such molecules combine the properties of RNAse catalytic activity and the ability to interact with specific sequences of complementary RNA targets.

According to the present invention an LNA modified oligonucleotide can act as a ribozyme to combine RNase catalytic activity with the ability to interact sequence specifically with a complementary RNA target. As used herein, ribozymes are intended to include RNA molecules that contain antisense sequences for specific sequence recognition, and an RNA-cleaving enzymatic activity. Ribozymes have been reported to be effective in cell cultures against viral targets. Oligonucleotides with an ACCA sequence at one end, referred to as “external guide sequences” (EGS's), were hybridized to a specific sequence on an RNA molecule. The RNA molecule with the bound EGS thereby becomes a substrate for RNase P and is specifically cleaved by RNase P.

The utility of an LNA-modified oligonucleotide for modulation (including inhibition) of expression of a targeted gene can be readily determined by simple testing. Thus, as discussed above, an in vitro or in vivo expression system comprising the targeted gene, mutations or fragments thereof, can be contacted with a particular LNA-modified oligonucleotide and levels of expression are compared to a control, that is, using the identical expression system which was not contacted with the LNA-modified oligonucleotide.

As used herein, the term “LNA-modified oligonucleotide” or simply “LNA oligonucleotide” includes any oligonucleotide either fully or partially modified with LNA monomers. Thus, an LNA-modified oligonucleotide may be composed entirely of LNA monomers, or an LNA-modified oligonucleotide may comprise at least about one LNA monomer. Typical LNA-olignucleotides will contain at least 4 nucleic acid units, more typically at least about 6, 8, 10, 12, 14, 16, 18, 20, 24, 28, 30, 34, 38, 40, 45, 50, 55 or 60 nucleic acids, with as at least or up to 1, 2, 3, 4, 5, 6, 7, 8, 9, 10, 11, 12, 13, 14, 15, 16, 17, 18, 19 or 20 LNA units. LNA-modified oligonucleotides having more than about 80, 90, 100 or 120 nucleic acid units are often less preferred, at least for some applications.

As used herein, the term “modulation of host immune functions” refers to fluctuations in the numbers of immune cells, fluctuations in the levels of humoral proteins and fluctuations in chemokine responses as compared to the levels of each of the above in a normal healthy individual.

As used herein, the term “DNA repair gene” refers to a gene that is part of a DNA repair pathway, that when altered, permits mutations to occur in the DNA of the organism.

As used herein, the singular forms “a”, “an” and “the” include plural referents unless the context clearly dictates otherwise.

As used herein, the term “infectious agent” refers to an organism wherein growth/multiplication leads to pathogenic events in humans or animals. Examples of such agents are: bacteria, fungi, protozoa and viruses.

As used herein, the term “oligonucleotide specific for” refers to an oligonucleotide having a sequence (i) capable of forming a stable complex with a portion of the targeted gene, e.g. by either strand invasion or triplex formation, a mechanism also called antigene or (ii) capable of forming a stable duplex with a portion of a mRNA transcript of the targeted gene a mechanism also called antisense.

As used herein, “portion of a sequence” refers to the minimum number of bases in a target sequence to which an LNA-modified oligonucleotide binds to and can modulate the activity of that target sequence. The “activity” of the target sequence can be any step that is involved in the pathogenic mechanism of a particular infectious agent. For example, the LNA-modified oligonucleotide can bind to nucleic acid sequences that code for the ligand which allows attachment and/or entry of the infectious agent into a host cell, thereby inhibiting the synthesis of such a ligand.

As used herein, the term “oligonucleotide” includes linear or circular oligomers of natural and/or modified monomers or linkages, including deoxyribonucleosides, ribonucleosides, substituted and alpha-anomeric forms thereof, peptide nucleic acids (PNA), and the like, capable of specifically binding to a target polynucleotide by way of a regular pattern of monomer-to-monomer interactions, such as Watson-Crick type of base pairing, Hoogsteen or reverse Hoogsteen types of base pairing, or the like.

The oligonucleotide may be composed of a single region or may be composed of several regions. The oligonucleotide may be “chimeric”, that is, composed of different regions. In the context of this invention “chimeric” antisense compounds are antisense compounds, particularly oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically are comprised of at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. One ore more regions of the oligonucleotide may serve as a substrate for enzymes capable of cleaving RNA:DNA or RNA:RNA hybrids. There are several enzymes with such catalytic effect. A method of digesting RNA at a specific location with an antisense oligonucleotide and an RNase H has been demonstrated by Minshull et al. (Nucleic Acids Research, 14:6433-6451 (1986)). Rnase H is a cellular endonuclease which cleaves the RNA strand of an RNA:DNA duplex. Therefore, activation of RNase H results in cleavage of the RNA target. The efficiency of oligonucleotide inhibition of gene expression might therefore be enhanced. Other enzymes capable of cleaving are Rnase L and Rnase P.

The chimeric oligonucleotides or antisense compounds of the present invention can be formed as mixed structures of two or more oligonucleotides, modified oligonucleotides, oligonucleosides and/or oligonucleotide analogs as described above. These include a type wherein the “gap” region of linked nucleosides is positioned between 5′ and 3′ segments of linked nucleosides. A second “open end” type wherein the “gap” region is located at either the 3′ or the 5′ terminus of the oligomeric compound. Oligonucleotides of the first type are also known in the art as “hybrids”, “gapmers” or gapped oligonucleotides. Oligonucleotides of the second type are also known as “wingmers” or “tailmers”.

The oligonucleotide can be composed of regions that can be linked in “register”, that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “monomers” typically indicates monomers linked by phosphodiester bonds or analogs thereof to form oligolnucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139(1999); Freier S., M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000),); 2′-O, 3′-C-linked [3.2.0] bicycloarabinonucleosides (see e.g. N. K Christiensen., et al, J. Am. Chem. Soc., 120: 5458-5463 (1998). Such analogs include synthetic nucleosides designed to enhance binding properties, e.g., duplex or triplex stability, specificity, or the like.

The term “stability” in reference to duplex or triplex formation generally designates how tightly an antisense oligonucleotide binds to its intended target sequence; more particularly, “stability” designates the free energy of formation of the duplex or triplex under physiological conditions. Melting temperature under a standard set of conditions, e.g., as described below, is a convenient measure of duplex and/or triplex stability. Preferably, antisense oligonucleotides of the invention are selected that have melting temperatures of at least 45° C. when measured in 100 mM NaCl, 0.1 mM EDTA and 10 mM phosphate buffer aqueous solution, pH 7.0 at a strand concentration of both the antisense oligonucleotide and the target nucleic acid of 1.5 μM. Thus, when used under physiological conditions, duplex or triplex formation will be substantially favored over the state in which the antisense oligonucleotide and its target are dissociated. It is understood that a stable duplex or triplex may in some embodiments include mismatches between base pairs and/or among base triplets in the case of triplexes. Preferably, LNA modified antisense oligonucleotides of the invention form perfectly matched duplexes and/or triplexes with their target nucleic acids.

As used herein, the term “downstream” when used in reference to a direction along a nucleotide sequence means in the direction from the 5′ to the 3′ end. Similarly, the term “upstream” means in the direction from the 3′ to the 5′ end.

As used herein, the term “gene” means the gene and all currently known variants thereof and any further variants which may be elucidated.

As used herein, the term mRNA means the presently known mRNA transcript(s) of a targeted gene, and any further transcripts which may be elucidated.

In the present context, the term “photochemically active groups” refers to compounds which are able to undergo chemical reactions upon irradiation with light. Illustrative examples of functional groups herein are quinones, especially 6-methyl-1,4-naphtoquinone, anthraquinone, naphtoquinone, and 1,4-dimethyl-anthraquinone, diazirines, aromatic azides, benzophenones, psoralens, diazo compounds, and diazirino compounds.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the LNA oligonucleotide has at least 80% or 90%, preferably 95%, most preferably 100%, complementarity to a defined sequence. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

The LNA modified antisense oligonucleotide (vide infra) is administered to a patient by any of the routes described hereinafter.

LNA modified antisense oligonucleotides may be used in combinations. For instance, a cocktail of several different LNA modified oligonucleotides, directed against different regions of the same gene, may be administered simultaneously or separately.

According to one preferred embodiment of the invention, the LNA-modified antisense oligonucleotide is specific for genes responsible for viral replication; viral infection cycle such as attachment to cellular ligands; viral genes encoding host immune modulating functions. Examples of) viral organisms include, but not restricted to, those listed in table 1. For information about the viral organisms see Fields of Virology, 3. ed., vol 1 and 2, BN Fields et al. (eds.). Non-limiting examples of targets of selected viral organisms are listed in table 2.

TABLE 1
Selected viral organisms causing human diseases.

Herpesviruses
Alpha-herpesviruses:
Herpes simplex virus 1 (HSV-1)
Herpes simplex virus 2 (HSV-2)
Varicella Zoster virus (VZV)
Beta-herpesviruses:
Cytomegalovirus (CMV)
Herpes virus 6 (HHV-6)
Gamma-herpesviruses:
Epstein-Barr virus (EBV)
Herpes virus 8 (HHV-8)
Hepatitis viruses
Hepatitis A virus
Hepatitis B virus
Hepatitis C virus (see Example 4)
Hepatitis D virus
Hepatitis E virus
Retroviruses
Human Immunodeficiency 1 (HIV-1)(see
Example 3)
Orthomyxoviruses
Influenzaviruses A, B and C
Paramyxoviruses
Respiratory Syncytial virus (RSV)
Parainfluenza viruses (PI)
Mumps virus
Measles virus
Togaviruses
Rubella virus
Picornaviruses
Enteroviruses
Rhinoviruses
Coronaviruses
Papovaviruses
Human papilloma viruses (HPV)
Polyomaviruses (BKV and JCV)
Gastroenteritisviruses

TABLE 2
Target genes of selected viral organisms

| | | open reading | |
|---|---|---|---|
| Organism | target gene | frame | gene product |
| HIV | gag: | MA | p17 |
| | | CA | p24 |
| | | NC | p7 |
| | | | p6 |
| | pol: | PR | p15 |
| | | RT | p66 |
| | | | p31 |
| | env: | | gp120 |
| | | | gp41 |
| | tat | | transcriptional transactivator |
| | rev | | regulator of viral expression |
| | vif | | |
| | vpr | | |
| | vpu | | |
| | nef | | |
| RSV | NS1 | | |
| | NS2 | | |
| | L | | |
| | 2-5A- | | |
| | dependent | | |
| | Rnase L | | |
| HPV | E1 | | helicase |
| | E2 | | transcription regulator |
| | E3 | | |
| | E4 | | late NS protein |
| | E5 | | transforming protein |
| | E6 | | transforming protein |
| | E7 | | transforming protein |
| | E8 | | |
| | L1 | | major capsid protein |
| | L2 | | minor capsid protein |
| HCV | NS3 | | protease |
| | NS3 | | helicase |
| | HCV-IRES | | (see Example 4) |
| | NS5B | | polymerase |
| HCMV | DNA | | |
| | polymerase | | |
| | IE1 | | |
| | IE2 | | |
| | UL36 | | |
| | UL37 | | |
| | UL44 | | polymerase ase. protein |
| | UL54 | | polymerase |
| | UL57 | | DNA binding protein |
| | UL70 | | primase |
| | UL102 | | primase asc. protein |
| | UL112 | | |
| | UL113 | | |
| | IRS1 | | |
| VZV | | 6 | |
| | | 16 | |
| | | 18 | |
| | | 19 | |
| | | 28 | |
| | | 29 | |
| | | 31 | |
| | | 39 | |
| | | 42 | |
| | | 45 | |
| | | 47 | |
| | | 51 | |
| | | 52 | |
| | | 55 | |
| | | 62 | |
| | | 71 | |
| HSV | IE4 | | US1 |
| | IE5 | | US12 |
| | IE110 | | ICP0 |
| | IE175 | | ICP4 |
| | UL5 | | helicase |
| | UL8 | | helicase |
| | UL13 | | capsid protein |
| | UL30 | | polymerase |
| | UL39 | | ICP6 |
| | UL42 | | DNA binding protein |

Information about the above selected genes, open reading frames and gene products is found in the following references: Field A. K. and Biron, K. K. “The end of innocence” revisited: resistance of herpesviruses to antiviral drugs. Clin. Microbiol. Rev. 1994; 7: 1-13. Anonymous. Drug resistance in cytomegalovirus: current knowledge and implications for patient management. J. Acquir. Immune Defic. Syndr. Hum. Retrovir. 1996; 12: S1-SS22. Kelley R et al.. Varicella in children with perinatally acquired human immunodeficiency virus infection. J Pediatr 1994; 124: 271-273. Hanecak et al. Antisense oligonucleotides inhibition of hepatitis C virus. gene expression in transformed hepatocytes. J Virol 1996; 70: 5203-12. Walker Drug discovery Today 1999; 4: 518-529. Zhang et al. Antisense oligonucleotides inhibition of hepatitis C virus (HCV) gene expression in livers of mice infected with an HCV-Vaccinia virus recombinant. Antim. Agents Chemotherapy 1999; 43, 347-53. Feigin R D, Cherry J D, eds. Textbook of pediatric infectious diseases. Philadelphia: W B Saunders, 1981. Chen B.et al., Induction of apoptosis of human cervical carcinoma cell line SiHa by antisense oligonucleotide og human papillomavirus type 16 E6 gene. 2000; 21(3): 335-339. The human herpesviruses. New York: Raven Press; 1993. DeClerque E, Walker R T, eds. Antiviral drug development: a multi-disciplinary approach. Plenum; 1987. Antiviral Drug Resistance (Richman, D. D., ed.), Wiley, Chichester, 1995. Flint S J et al. eds. Principles of virology: Molecular biology, pathogenesis and control.

It should be appreciated that in the above table 2, an indicated gene means the gene and all currently known variants thereof, including the different mRNA transcripts that the gene and its variants can give rise to, and any further gene variants which may be elucidated. In general, however, such variants will have significant sequence identity to a sequence of table 2 above, e.g. a variant will have at least about 70 percent sequence identity to a sequence of the above table 2, more typically at least about 75, 80, 85, 90, 95, 97, 98 or 99 percent sequence identity to a sequence of the above table 2. Sequence identity of a variant can be determined by any of a number of standard techniques such as a BLAST program http://www.ncbi.nlm.nih.gov/blast/).

Sequences for the genes listed in Table 2 can be found in GenBank (http://www.ncbi.nlm.nih.gov/). The gene sequences may be genomic, cDNA or mRNA sequences. Preferred sequences are viral genes containing the complete coding region and 5′ untranslated sequences that are involved in viral replication.

In vitro propagation of virus causing human diseases: To screen for antiviral effect of antisense oligonucleotides viral particles are propagated in in vitro culture systems of appropriate mammalian cells. Initial screening is typically performed in transformed cell lines. More thorough screening is typically performed in human diploid cells.

TABLE 3
Examples of in vitro propagation of viruses.

| Organism | WI-38 or MRC-5 | HeLa or HEp-2 | PRMK or PCMK |
|---|---|---|---|
| HSV | C, D, S | D | D |
| HCMV | C, F | — | — |
| VZV | C, F | — | — |
| Adeno | D | D | D |
| RSV | S | S | S |
| Polio | D | D | D |
| Echo | D | — | D |
| Rhino | D, F | — | D, F |

C is cytomegaly, D is cell destruction, F is marked focality, H is hemadsorption and S is formation of syncytium. “-” means that the cell line does not sustain growth of the virus. WI-38 is a human diploid fibroblast cell line. MRC-5 is human lung fibroblasts. HeLa is a human aneuploid epithelial cell line. PRMK is primary rhesus monkey kidney cells. PCMK is primary cynomolgus monkey kidney cells.

Likewise Vero cells (green monkey kidney cells) and Mewo cells will sustain the growth of for example herpesviruses. References: DeClerque E, Walker R T, eds. Antiviral drug development: a multi-disciplinary approach. Plenum; 1987. Antiviral Drug Resistance (Richman, D. D., ed.), Wiley, Chichester, 1995. Cytomegalovirus protocols, J. Sinclair (ed.), Humana Press. HIV Protocols, N. Michael and J H Kim (eds.), Humana press. Hepatitis C Protocols, J Y N Lau (ed.), Humana Press. Antiviral Methods and Protocols, D Kinchington and R F Schinazi, Humana Press.

Bacterial infections: According to another preferred embodiment of the invention, the LNA-modified antisense oligonucleotide is specific for the human or domestic animal bacterial pathogens listed in (but not restricted to) table 4. The target genes may include (but are not restricted to) genes essential to bacterial survival and multiplication in the host organism, virulence genes, genes encoding single- or multi-drug resistance such as for instance the genes listed in table 5.

TABLE 4
Selected bacteria causing serious human diseases

Gram positive organisms:
Staphylococcus aureus: strains include
methicillin resistant (MRSA), methicillin-
vancomycin resistant (VMRSA) and
vancomycin intermediate resistant (VISA).
Staphylococcus epidermidis.
Enterococcus faecalis and E. fuecium: strains
include vancomycin resistant (VRE).
Streptococcus pneumoniae.
Gram negative organisms:
Pseudomonas aeruginosa.
Burkholdia cepacia.
Xanthomonas maltophila.
Escherichia coli
Enterobacter spp.
Klebsiella pneumoniae
Salmonella spp.

References: Cookson B. D., Nosocomial antimicrobial resistance surveillance. J. Hosp. Infect. 1999:97-103. Richards M. J. et al.. Nosocomial infections in medical intensive care units in the United States. National Nosocomial Infections Surveillance System. Crit. Care. Med. 1999;5:887-92. House of Lords Select Committee on Science and Technology. Resistance to antibiotics and other antimicrobial agents. London: 1998; Her Majesty's Stationary Office. Johnson A. P.. Intermediate vancomycin resistance in S. aureus: a major threat or a minor inconveniance? J. Antimicrobial. Chemother. 1998;42:289-91. Baquero F.. Pneumococcal resistance to beta-lactam antibiotics: a global overview. Microb. Drug Resist. 1995;1:115-20. Hsueh P. R. et al.. Persistence of a multidrug resistant Pseudomonas aeruginosa clone in an intensive care burn unit. J. Clin. Microbiol. 1998;36:1347-51. Livermore D.. Multiresistance and Superbugs. Commun. Dis. Public Health 1998;1:74-76.

The preferred antisense target genes in bacteria would include (but are not restricted to) genes involved in the following biological functions: 1. Protein synthesis; 2. Cell wall synthesis; 3: Cell division; 4: Nucleic acid synthesis; and 5: Virulence. The biological functions mentioned are analogous in Gram positive and Gram negative bacteria, and the genes encoding the individual proteins involved may exhibit extensive homologies in their nucleotide sequences. The genes encoding the mentioned target complexes may have different names in different bacteria.

TABLE 5
Examples of selected antisense target complexes in bacteria.

| | |
|---|---|
| Protein synthesis | Translation initiation factors (e.g. IF1, IF2, IF3) |
| targets | |
| | Translation elongation factors (e.g. EF-Tu, EF-G) |
| | Translation release factors (RF1, RF2, RF3) |
| Cell wall synthesis | Penicillin binding proteins (e.g. PBP1 to PBP9) |
| Cell division | Proteins encoded by the ftsQAZ operon |
| Nucleic acid synthesis | Gyrases, Sigma 70 and Helicase |
| Virulence | Ureases |

References: Escherichia coli and Salmonella in Cellular and Molecular Biology, vol 1 & 2. C Neidhardt and R Curtiss (eds.), American Society for Microbiology Press. Gram-Positive Pathogens. V A Fischetti et al. (eds.), American Society for Microbiology Press. Bacterial Pathogenesis: A Molecular Approach. A A Salyers and D D Whitt (eds.), American Society for Microbiology Press. Organization of the Procaryotic Genome. R L Charlebois (ed.), American Society for Microbiology Press.

Listed in Table 6 below are examples of genes encoding the protein complexes listed in Table 5 above. The individual genes have homologues in the major human pathogenic bacteria listed in Table 4. Table 6 below depicts an example of a Gram negative (Escherichia coli) and a Gram positive (Staphylococcus aureus) organism, chosen as representatives for the two groups of bacteria.

TABLE 6
Examples of genes encoding possible antisense target proteins.

| Target group | E. coli | S. aureus |
|---|---|---|
| Protein synthesis | prfA | prfA |
| | prfB | |
| | prfC | prfC |
| | infA | infA |
| | infB | infB |
| | infC | |
| | tufA | tuf |
| | fusA | fus |
| Cell wall synthesis | mrcA | pbpA |
| | mrcB | pbp2 |
| | pbpB | fmhB |
| | | femA |
| | | femB |
| Cell division | ftsA | ftsA |
| | ftsQ | |
| | ftsZ | ftsZ |
| Nucleic acid synthesis | gyrA | pcrC |
| | gyrB | |
| | rpoD | |

References: Escherichia coli and Salmonella in Cellular and Molecular Biology, vol 1 & 2. C Neidhardt and R Curtiss (eds.), American Society for Microbiology Press. Gram-Positive Pathogens. V A Fischetti et al. (eds.), American Society for Microbiology Press. Bacterial Pathogenesis: A Molecular Approach. A A Salyers and D D Whitt (eds.), American Society for Microbiology Press. Organization of the Procaryotic Genome. R L Charlebois (ed), American Society for Microbiology Press.

Related bacterial species among the Gram negatives as well as the Gram positives exhibit homologous genes that serve as antisense targets.

It should be appreciated that in the above table 5 and 6, an indicated gene means the gene and all currently known variants thereof, including the different mRNA transcripts that the gene and its variants can give rise to, and any further gene variants which may be elucidated. In general, however, such variants will have significant sequence identity to a sequence of table 5 and 6 above, e.g. a variant will have at least about 70 percent sequence identity to a sequence of the above table 5 and 6, more typically at least about 75, 80, 85, 90, 95, 97, 98 or 99 percent sequence identity to a sequence of the above table 5 and 6. Sequence identity of a variant can be determined by any of a number of standard techniques such as a BLAST program http://www.ncbi.nlm.nih.gov/blast/).

Sequences for the genes listed in Table 5 and 6 can be found in GenBank (http://www.ncbi.nlm.nih.gov/). The gene sequences may be genomic, cDNA or mRNA sequences. Preferred sequences are viral genes containing the complete coding region and 5′ untranslated sequences that are involved in viral replication.

LNA modified antisense oligonucleotides may be used in combinations. For instance, a cocktail of several different LNA modified oligonucleotides, directed against different regions of the same gene, may be administered simultaneously or separately.

Protozoan infections: According to one preferred embodiment of the invention, the LNA-modified antisense oligonucleotide is specific for protozoan organisms infecting humans and causing human diseases. Such protozoa include, but are not restricted to, the following: 1. Malaria e.g. Plasmodium falciparum and M. ovale. (references: Malaria by M Wahlgren and P Perlman (eds.), Harwood Academic Publishers, 1999. Molecular Immunological Considerations in Malaria Vaccine Development by M F Good and A J Saul, CRC Press 1993). 2. Trypanosomiasis (sleeping sickness) e.g. Trypanosoma cruzei (reference: Progress in Human African Trypanosomiasis, Sleeping Sickness by M Dumas et al. (eds.), Springer Verlag 1998). 3. Leischmaniasis e.g. Leiscimania donovani (reference: A L Banuals et al., Molecular Epidemiology and Evolutionary Genetics of Leischmania Parasites. Int J Parasitol 1999;29:1137-47). 4. Amebiasis e.g. Entamoeba histolytica (R P Stock et al., Inhibition of Gene Expression in Entamoeba histolytica with Antisense Peptide Nucleic Acid Oligomers. Nature Biotechnology 2001;19:231-34).

Fungal infections: According to one preferred embodiment of the invention, the LNA-modified antisense oligonucleotide is specific for fungi cause pathogenic infections in humans. These include, but are not restricted to, the following: Candida albicans (references: A H Groll et al., Clinical pharmacology of systemic antifungal agents: a comprehensive review of agents in clinical use, current investigational compounds, and putative targets for antifungal drug development. Adv. Pharmacol. 1998:44:343-501. M D D Backer et al., An antisense-based functional genomics approach for identification of genes critical for growth of Candida albicans. Nature Biotechnology 2001;19:235-241) and others, e.g., Histoplasma neoformans, Coccidioides immitis and Penicillium marneffei (reference: S A Marques et al., Mycoses associated with AIDS in the Third World. Med Mycol 2000;38 Suppl 1:269-79).

Host cellular genes involved in viral diseases: According to one preferred embodiment of the invention, the LNA-modified antisense oligonucleotide is specific for host cellular genes involved in viral diseases. Besides genes encoded by viruses for their replication, the initial step to infection is binding to cellular ligands. For example CD4, chemokine receptors such as CCR3, CCR5 are required for HIV infection. Furthermore, viruses also upregulate certain chemokines which aid in their replication, for example in the case of HIV there is an increase in IL-2 which results in an increase of CD4⁺T cells. allowing for an increase in the pool of cells for further infection in the early stages of the disease. The LNA modified antisense oligonucleotides may be used to prevent any further upregulation of genes that may aid in the infectivity and replication rate of the viruses. Preferred targets are the 5′ untranslated sequences of ligands used by viruses to infect a cell, or any other cellular factor that aids in the replication of the viruses. Particularly preferred are human cDNA sequences. According to the invention LNA modified oligonucleotides may be used to modulate the expression of genes involved in the viral infection cycle.

LNA modified oligonucleotides against genes involved in infectious diseases caused by viruses, bacteria, protozoa, fungi, parasites, etc., may be used in combinations. For instance, a cocktail of several different LNA modified oligonucleotides, directed against different regions of the same gene, may be administered simultaneously or separately. Also, combinations of LNA modified antisense oligonucleotides specific for different genes, such as for instance the HBV P, S, and C gene, may be administered simultaneously or separately. LNA modified oligonucleotides may also be administered in combination with other antiviral drugs, antibiotics, etc.

In the practice of the present invention, target genes may be single-stranded or double-stranded DNA or RNA; however, single-stranded DNA or RNA targets are preferred. It is understood that the target to which the antisense oligonucleotides of the invention are directed include allelic forms of the targeted gene and the corresponding mRNAs including splice variants. There is substantial guidance in the literature for selecting particular sequences for antisense oligonucleotides given a knowledge of the sequence of the target polynucleotide, e.g., Cook S. T. Antisense Drug Technology, Principles, Strategies, and Applications, Marcel Dekker, Inc, 2001; Peyman and Ulmann, Chemical Reviews, 90:543-584, 1990; and Crooke, Ann. Rev. Pharmacol. Toxicol., 32:329-376 (1992). Preferred mRNA targets include the 5′ cap site, tRNA primer binding site, the initiation codon site, the mRNA donor splice site, and the mRNA acceptor splice site.

Where the target polynucleotide comprises a mRNA transcript, sequence complementary oligonucleotides can hybridize to any desired portion of the transcript. Such oligonucleotides are, in principle, effective for inhibiting translation, and capable of inducing the effects described herein. It is hypothesized that translation is most effectively inhibited by blocking the mRNA at a site at or near the initiation codon. Thus, oligonucleotides complementary to the 5′-region of mRNA transcript are preferred. Oligonucleotides complementary to the mRNA, including the initiation codon (the first codon at the 5′ end of the translated portion of the transcript), or codons adjacent to the initiation codon, are preferred.

While antisense oligomers complementary to the 5′-region of the mRNA transcripts are preferred, particularly the region including the initiation codon, it should be appreciated that useful antisense oligomers are not limited to those oligomers complementary to the sequences found in the translated portion of the mRNA transcript, but also includes oligomers complementary to nucleotide sequences contained in, or extending into, the 5′- and 3-untranslated regions. Antisense oligonucleotides complementary to the 3′-untranslated region may be particularly useful in regard to increasing the half-life of a mRNA thereby potentially up-regulating its expression.

It is well known that many sequences in a mRNA cannot be addressed by standard oligonucleotides employing oligonucleotides of moderate affinity, e.g., oligonucleotides composed of DNA and/or RNA monomers or the currently used analogues. It is believed that this problem is primarily due to intra-molecular base-pairings structures in the target mRNA. The use of appropriately designed LNA modified oligonucleotides can effectively compete with such structures due to the increased affinity of such oligonucleotides compared to the unmodified reference oligonucleotides. Thus, LNA can be used to design antisense oligonucleotides with a greater therapeutic potential than that of current antisense oligonucleotides.

LNA modified antisense oligonucleotides of the invention can comprise any polymeric compound capable of specifically binding to a target oligonucleotide by way of a regular pattern of monomer-to-monomer interactions, such as Watson-Crick type of base pairing, Hoogsteen or reverse Hoogsteen types of base pairing, or the like. An LNA modified antisense oligonucleotide will have higher affinity for the target sequence compared with the corresponding unmodified reference oligonucleotide of similar sequence.

As used herein, the term “corresponding unmodified reference oligonucleotide” refers to an oligonucleotide solely consisting of naturally occurring nucleotides that represent the same nucleobase sequence in the same orientation as the modified oligonucleotide.

A particular aspect of the invention is the use of LNA monomers to enhance the potency, specificity and duration of action and broaden the routes of administration of oligonucleotides comprised of current chemistries such as MOE, ANA, FANA, PS etc (ref: Recent advances in the medical chemistry of antisense oligonucleotide by Uhlman, Current Opinions in Drug Discovery & Development 2000 Vol 3 No 2). This can be achieved by substituting some of the monomers in the current oligonucleotides by LNA monomers. The LNA modified oligonucleotide may have a size similar to the parent compound or may be larger or preferably smaller. It is preferred that such LNA-modified oligonucleotides contain less than about 70%, more preferably less than about 60%, most preferably less than about 50% LNA monomers and that their sizes are between about 10 and 25 nucleotides, more preferably between about 12 and 20 nucleotides.

A further aspect of the invention is the use of different LNA monomers in the oligonucleotide such as for example the oxy-LNA, thio-LNA or amino-LNA monomers.

The use of such different monomers offers a means to “fine tune” the chemical, physical, biological, pharmacokinetic and pharmacological properties of the oligonucleotide thereby facilitating improvement in their safety and efficacy profiles when used as antisense drugs.

An “LNA modified oligonucleotide” or “LNA oligonucleotide” is preferably used herein to describe oligonucleotides comprising at least one LNA monomeric residue of the following Formula I:

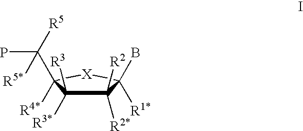

wherein in that Formula I: X is selected from —O—, —S—, —N(R^{N})—, —C(R⁶R^{6*})—, —O—C(R⁷R^{7*})—, —C(R⁶R^{6*})—O—, —S—C(R⁷R^{7*})—, —C(R⁶R^{6*})—S—, —N(R^{N*})—C(R⁷R^{7*})—, —C(R⁶R^{6*})—N(R^{N*})—, and —C(R⁶R^{6*})—C(R⁷R^{7*})—;

B is selected from hydrogen, hydroxy, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands;

P designates the radical position for an internucleoside linkage to a succeeding monomer, or a 5′-terminal group, such internucleoside linkage or 5′-terminal group optionally including the substituent R⁵;

one of the substituents R², R^{2*}, R³, and R^{3*} is a group P* which designates an internucleoside linkage to a preceding monomer, or a 2′/3′-terminal groups;

the substituents of R^{1*}, R^{4*}, R⁵, R^{5*}, R⁶, R^{6*}, R⁷, R^{7*}, R^{N}, and the ones of R², R^{2*}, R³, and R^{3*} not designating P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C═Q,

wherein Q is selected from —O—, —S—, and —N(R^{a})—, and R^{a} and R^{b} each is independently selected from hydrogen, optionally substituted C₁₋₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R^{1*}, R², R^{2*}, R³, R^{3*}, R^{4*}, R⁵, R^{5*}, R⁶ and R^{6*}, R⁷, and R^{7*} which are present and not involved in P, P* or the biradical(s) together may form an associated biradical selected from biradicals of the same kind as defined before; said pair(s) of non-geminal substituents thereby forming a mono- or bicyclic entity together with (i) the atoms to which said non-geminal substituents are bound and (ii) any intervening atoms; and

each of the substituents R^{1*}, R², R^{2*}, R³, R^{4*}, R⁵, R^{5*}, R⁶ and R^{6*}, R⁷, and R^{7*} which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —N(R^{N})— where RN is selected from hydrogen and C₁₋₄-alkyl, and where two adjacent (non-geminal) substituents may designate an additional bond resulting in a double bond; and R^{N*}, when present and not involved in a biradical, is selected from hydrogen and C₁₋₄-alkyl; and basic salts and acid addition salts thereof;

In another preferred embodiment, LNA modified oligonucleotides used in this invention comprises oligonucleotides containing at least one LNA monomeric residue of the Formula I above:

Wherein X, B, P are defined as above;

one of the substituents R², R^{2*}, R³, and R^{3*} is a group P* which designates an internucleoside linkage to a preceding monomer, or a 2′/3′-terminal group;

R¹-R7* substituent together designates a biradical structure selected from —(CR*R*)ᵣ—M—(CR*R*)ₛ—, —(CR*R*)ᵣ—M—(CR*R*)ₛ—M—, —M—(CR* R*)ᵣ₊ₛ—M—, —M—(CR*R*)ᵣ—M—(CR*R*)ₛ—, —(CR*R*)ᵣ₊ₛ—, —M—, —M—M—, wherein each M is independently selected from —O—, —S—, —Si(R*)₂—, —N(R*)—, >C═O, —C(═O)—N(R*)—, and —N(R*)—C(═O)—. Each R* and R^{1(1*)}—R^{7(7*)}, which are not involved in the biradical, are independently selected from hydrogen, halogen, azido, cyano, nitro, hydroxy, mercapto, amino, mono- or di(C₁₋₆-alkyl)amino, optionally substituted C₁₋₆-alkoxy, optionally substituted C₁₋₆-alkyl, DNA intercalators, photochemically active groups, thermochemically active groups, chelating groups, reporter groups, and ligands, and/or two adjacent (non-geminal) R* may together designate a double bond, and each of r and s is 0-4 with the proviso that the sum r+s is 1-5.

In a another preferred embodiment LNA modified oligonucleotides used in this invention comprises oligonucleotides containing at least one LNA monomeric residue of the general formula shown scheme II:

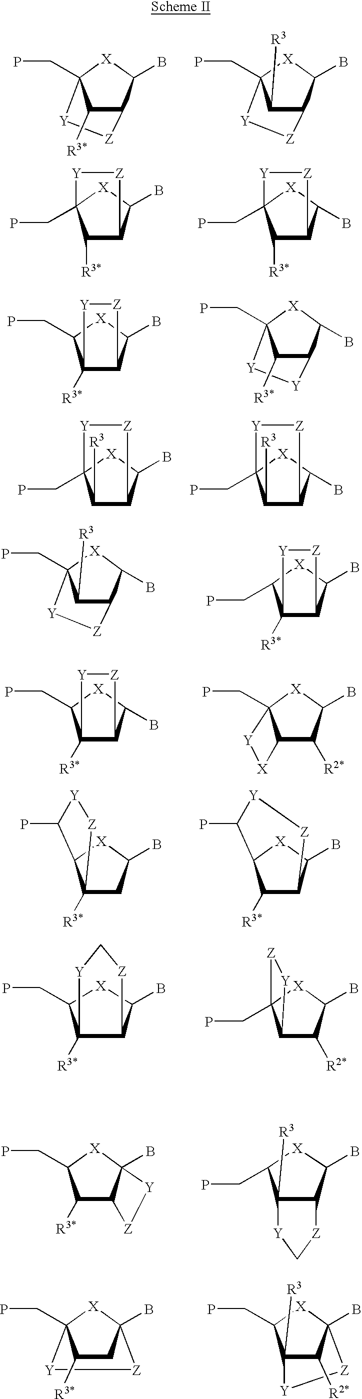

Wherein X and B are defined as above.

P designates the radical position for an internucleoside linkage to a succeeding monomer, or a 5′-terminal group, such internucleoside linkage or 5′- terminal group optionally including the substituent R⁵;

one of the substituents R², R^{2*}, R³, and R^{3*} is a group P* which designates an internucleoside linkage to a preceding monomer, or a 2′/3′-terminal group;

Y-Z denotes a biradical structure constituted of non-geminal substituents that are selected from —(CR*R*)ᵣ—M—(CR*R*)ₛ—, —(CR*R*)ₛ—M—(CR*R*)ₛ—M—, —M—(CR*R*)ᵣ₊ₛ—M—, —M—(CR*R*)ᵣ—M—(CR*R*)ₛ—, —(CR*R*)ᵣ₊ₛ—, —M—, —M—M—, wherein each M is independently selected from —O—, —S—, —Si(R*)₂—, —N(R*)—, >C═O, —C(═O)—N(R*)—, and —N(R*)—C(═O)—. Each R^{*} are independently selected from hydrogen, halogen, azido, cyano, nitro, hydroxy, mercapto, amino, mono- or di(C₁₋₃-alkyl)amino, optionally substituted C₁₋₃-alkoxy, optionally substituted C₁₋₆-alkyl, DNA intercalators, photochemically active groups, thermochemically active groups, chelating groups, reporter groups, and ligands, and/or two adjacent (non-geminal) R* may together designate a double bond, and each of r and s is 0-3 with the proviso that the sum r+s is 1-4.

In one embodiment of the invention, LNA monomer typically refers to a conformationally locked nucleoside having a 2′-4′ cyclic linkage, as described in the International Patent Application WO 99/14226 and subsequent applications, WO0056746, WO0056748, WO0066604, PA 2000 01473, DK PA 1999 00381, U.S. provisional No. 60/127,357 and DK PA 1999 00603, U.S. provisional No. 60/133,273, all incorporated herein by reference. Preferred LNA monomer structures are exemplified in the formulae Ia and Ib below. In formula Ia the configuration of the furanose is denoted D-β, and in formula Ib the configuration is denoted L-α. Configurations which are composed of mixtures of the two, e.g. D-α and L-β, are also included.

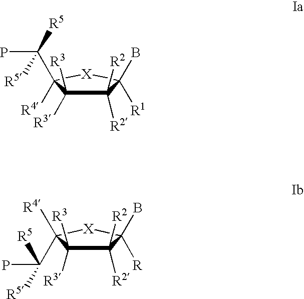

In Ia and Ib, X is oxygen, sulfur and carbon; B is a nucleobase, e.g. adenine, cytosine, 5-methylcytosine, isocytosine, pseudoisocytosine, guanine, thymine, uracil, 5-bromouracil, 5-propynyluracil, 5-propyny-6-fluoroluracil, 5-methylthiazoleuracil, 6-aminopurine, 2-aminopurine, inosine, diaminopurine, 7-propyne-7-deazaadenine, 7-propyne-7-deazaguanine. R¹, R² or R^{2′}, R³ or R^{3′}, R⁵ and R^{5′} are hydrogen, methyl, ethyl, propyl, propynyl, aminoalkyl, methoxy, propoxy, methoxy-ethoxy, fluoro, chloro.

P designates the radical position for an internucleoside linkage to a succeeding monomer, or a 5′-terminal group, R³ or R^{3′} is an internucleoside linkage to a preceding monomer, or a 3′-terminal group. The internucleotide linkage may be a phosphate, phosphorothioate, phosphorodithioate, phosphoramidate, phosphoroselenoate, phosphorodiselenoate, alkylphosphotriester, methyl phosphornates.

The internucleotide linkage may also contain non-phosphorous linkers, hydroxylamine derivatives (e.g. —CH₂—NCH₃—O—CH₂—), hydrazine derivatives, e.g. —CH₂—NCH₃—NCH₃—CH₂, amid derivatives, e.g. —CH₂—CO—NH—CH₂—, CH₂—NH—CO—CH₂—. In Ia, R^{4′} and R^{2′} together designate —CH₂—O—, —CH₂—S—, —CH₂—NH— or —CH₂—NMe— where the oxygen, sulfur or nitrogen, respectively, is attached to the 2′-position.

In Formula Ib, R^{4′} and R² together designate —CH₂—O—, —CH₂—S—, —CH₂—NH— or —CH₂—NMe— where the oxygen, sulphur or nitrogen, respectively, is attached to the 2-position (R² configuration).

The internucleoside linkage is selected from linkages consisting of 2 to 4, preferably 3, groups/atoms selected from —CH₂—, —O—, —S—, —NR^{H}—, >C═O, >C═NR^{H}, >C═S, —Si(R″)₂——SO—, —S(O)₂—, —P(O)₂—, —P(O,S)—, —P(S)₂—, —PO(R″)—, —PO(OCH₃)—, and —PO(NHR^{H})—, where R^{H} is selected form hydrogen and C₁₋₄-alkyl, and R″ is selected from C₁₋₆-alkyl and phenyl.

In a preferred embodiment the internucleoside linkage is selected from —CH₂—CH₂—CH₂—, —CH₂—CO—CH₂—, —CH₂—CHOH—CH₂—, —O—CH₂—O—, —O—CH₂—CH₂—, —O—CH₂—CH═, —CH₂—CH₂—O—, —NR^{H}—CH₂—CH₂—, —CH₂—CH₂—NR^{H—, —CH}₂—NR^{H—CH}₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═, —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}—, —O—CH₂—S—, —S—CH₂—O—, —CH₂—CH₂—S—, —O—CH₂—CH₂—S—, —S—CH₂—CH═, —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(BH₃)—O—, —O—PO(NHR^{N})—O—, —O—P(O)₂—NR^{H}—, —NR^{H}—P(O)₂—O—, —O—P(O,NR^{H})—O—, and —O—Si(R″)₂—O—.

In a most preferred embodiment the internucleoside linkages are selected from —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl.

Very most preferred LNA monomer structures are structures in which X is oxygen (Formulae Ia, Ib); B is adenine, cytosine, 5-methylcytosine, isocytosine, pseudoisocytosine, guanine, thymine, uracil, 5-bromouracil, 5-propynyluracil, 5-propynyl-6-fluoroluracil, 6-aminopurine, 2-aminopurine, inosine, 2,6-diaminopurine, 7-propynyl-7-deazaadenine, 7-propynyl-7-deazaguanine; R¹, R² or R^{2′}, R³ or R^{3′}, R⁵ and R⁵, are hydrogen; R³ or R^{3′} is an internucleoside linkage to a preceding monomer, or a 3′-terminal group. In Formula Ia, R^{4′} and R^{2′} together designate —CH₂—O—, —CH₂—S—, —CH₂—NH— or —CH₂—NMe— where the oxygen, sulphur or nitrogen, respectively, is attached to the 2′-position, and in Formula Ib, R^{4′} and R² together designate —CH₂—O—, —CH₂—S—, —CH₂—NH— or —CH₂—NMe— where the oxygen, sulphur or nitrogen, respectively, is attached to the 2′-position in the R² configuration. P is a phosphate, phosphorothioate, phosphorodithioate, phosphoramidate, and methyl phosphornates;

LNA-modified compounds of the invention may also contain pendent groups or moieties, either as part of or separate from the basic repeat unit of the polymer, to enhance specificity, improve nuclease resistance, delivery, cellular uptake, cell and organ distribution, in vivo transport and clearance or other properties related to efficacy and safety, e.g., cholesterol moieties, duplex intercalators such as acridine, poly-L-lysine, “end-capping” with one or more nuclease-resistant linkage groups such as phosphoromonothioate, and the like.

Many pendant groups or moieties, when attached to an oligonucleotide, decrease its affinity for its complementary target sequence. Because the efficacy of an antisense oligo depends to a significant extend on its ability to bind with high affinity to its target sequence, such as pendant groups or moieties, even though being potentially useful, are not suitable for use with oligonucleotides composed of standard DNA, RNA or other moderate affinity analogues. Incorporation of LNA monomers into such oligonucleotides can be used as a means to compensate for the affinity loss associated with such pendant groups or moieties. Thus, LNA offers a general means for extracting the benefits of affinity decreasing pendant groups or moieties.

Incorporation of LNA monomers into a standard DNA or RNA oligonucleotide increases resistance towards nucleases (endonucleases and exonucleases), the extent of which will depend on the number of LNA monomers used and their position in the oligonucleotide. Nuclease resistance of LNA-modified oligonucleotides can be further enhanced by providing nuclease-resistant internucleosidic linkages. Many such linkages are known in the art, e.g., phosphorothioate: Zon and Geiser, Anti-Cancer Drug Design, 6:539-568 (1991); U.S. Pat. Nos. 5,151,510; 5,166,387; and 5,183,885; phosphorodithioates: Marshall et al., Science, 259:1564-1570 (1993); Caruthers and Nielsen, International Patent Application PCT/US89/02293; phosphoramidates, e.g., —O₂P(═O)(NR), where R may be hydrogen or C1-C3 alkyl; Jager et al., Biochemistry, 27:7237-7246 (1988); Froehler et al., International application PCT/US90/03138; peptide nucleic acids: Nielsen et al., Anti-Cancer Drug Design, 8:53-63 (1993), International application PCT/EP92/01220; methylphosphonates: U.S. Pat. Nos. 4,507,433; 4,469,863; and U.S. Pat. No. 4,757,055; and P-chiral linkages of various types, especially phosphorothioates, Stec et al., European patent application 506,242 (1992) and Lesnikowski, Bioorganic Chemistry, 21:127-155 (1993). Additional nuclease resistant linkages include phosphoroselenoate, phosphorodiselenoate, alkylphosphotriester such as methyl- and ethylphosphotriester, carbonate such as carboxymethyl ester, carbamate, morpholino carbamate, 3′-thioformacetal, silyl such as dialkyl (C1-C6)- or diphenylsilyl, sulfamate ester, and the like. Such linkages and methods for introducing them into oligonucleotides are described in many references, e.g., reviewed generally by Peyman and Ulmann, Chemical Reviews 90:543-584 (1990); Milligan et al., J. Med. Chem., 36:1923-1937 (1993); Matteucci et al., International application PCT/US91/06855; Toulmé, J. J., Nature Biotechnology 19:17-18 (2001); Manoharan M., Biochemica et Biophysica Acta 1489:117-139(1999); Freier S., M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000), ); 2′-O, 3′-C-linked [3.2.0] bicycloarabinonucleosides (see e.g. N. K Christiensen., et al, J. Am. Chem. Soc., 120: 5458-5463 (1998).

Resistance to nuclease digestion may also be achieved by modifying the internucleotide linkage at both the 5′ and 3′ termini with phosphoroamidites according to the procedure of Dagle et al., Nucl. Acids Res. 18, 4751-4757 (1990).

Preferably, phosphorus analogs of the phosphodiester linkage are employed in the compounds of the invention, such as phosphorothioate, phosphorodithioate, phosphoramidate, or methylphosphonate. More preferably, phosphoromonothioate is employed as the nuclease resistant linkage.

It is understood that in addition to the preferred linkage groups, compounds of the invention may comprise additional modifications, e.g., boronated bases, Spielvogel et al., U.S. Pat. No. 5,130,302; cholesterol moieties, Shea et al., Nucleic Acids Research, 18:3777-3783 (1990) or Letsinger et al., Proc. Natl. Acad. Sci., 86:6553-6556 (1989); and 5-propynyl modification of pyrimidines, Froehler et al., Tetrahedron Lett., 33:5307-5310 (1992).

In embodiments where triplex formation is desired, there are constraints on the selection of target sequences. Generally, third strand association via Hoogsteen type of binding is most stable along homopyrimidine-homopurine tracks in a double stranded target. Usually, base triplets form in T-A*T or C-G*C motifs (where “-” indicates Watson-Crick pairing and “*” indicates Hoogsteen type of binding); however, other motifs are also possible. For example, Hoogsteen base pairing permits parallel and antiparallel orientations between the third strand (the Hoogsteen strand) and the purine-rich strand of the duplex to which the third strand binds, depending on conditions and the composition of the strands. There is extensive guidance in the literature for selecting appropriate sequences, orientation, conditions, nucleoside type (e.g., whether ribose or deoxyribose nucleosides are employed), base modifications (e.g., methylated cytosine, and the like) in order to maximize, or otherwise regulate, triplex stability as desired in particular embodiments, e.g., Roberts et al., Proc. Natl. Acad. Sci., 88:9397-9401 (1991); Roberts et al., Science, 58:1463-1466 (1992); Distefano et al., Proc. Natl. Acad. Sci., 90:1179-1183 (1993); Mergny et al., Biochemistry, 30:9791-9798 (1992); Cheng et al., J. Am. Chem. Soc., 114:4465-4474 (1992); Beal and Dervan, Nucleic Acids Research, 20:2773-2776 (1992); Beal and Dervan, J. Am. Chem. Soc., 114:4976-4982; Giovannangeli et al., Proc. Natl. Acad. Sci., 89:8631-8635 (1992); Moser and Dervan, Science, 238:645-650 (1987); McShan et al., J. Biol. Chem., 267:5712-5721 (1992); Yoon et al., Proc. Natl. Acad. Sci., 89:3840-3844 (1992); and Blume et al., Nucleic Acids Research, 20:1777-1784 (1992).

The length of the oligonucleotide moieties is sufficiently large to ensure that specific binding takes place only at the desired target polynucleotide and not at other fortuitous sites, as explained in many references, e.g., Rosenberg et al., International application PCT/US92/05305; or Szostak et al., Meth. Enzymol, 68:419-429 (1979). The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, whether modifications to enhance binding or specificity are present, whether duplex or triplex binding is desired, and the like. Usually, antisense compounds of the invention have lengths in the range of about 8 or 12 to 40 nucleotides. More preferably, up to about 30 nucleotides; and most preferably, they have lengths in the range of about 8 or 12 to 20 or 30 nucleotides.

In general, the LNA-modified oligonucleotides used in the practice of the present invention have a sequence which is completely complementary to a selected portion of the target polynucleotide. Absolute complementarity, however, is not required, particularly in larger oligomers. Thus, reference herein to an “LNA-modified oligonucleotide sequence complementary to” a target polynucleotide does not necessarily mean a sequence having 100% complementarity with the target segment. In general, any oligonucleotide having sufficient complementarity to form a stable duplex with the target (e.g. a gene or its mRNA transcript) that is, an oligonucleotide which is “hybridizable”, is suitable. Stable duplex formation depends on the sequence and length of the hybridizing oligonucleotide and the degree of complementarity with the target polynucleotide. Generally, the larger the hybridizing oligomer, the more mismatches may be tolerated. More than one mismatch will probably not be tolerated for antisense oligomers of less than about 11 nucleotides. One skilled in the art may readily determine the degree of mismatching which may be tolerated between any given antisense oligomer and the target sequence, based upon the melting point, and therefore the thermal stability, of the resulting duplex. In general, an LNA-modified oligonucleotide will be at least about 60% complementary to a selected portion of the target polynucleotide, more typically an LNA-modified oligonucleotide will be at least about 70, 80, 90 or 95 percent complementary to a selected portion of the target polynucleotide.

The ability of an LNA-modified oligonucleotide to hybridize to a target polynucleotide also can be readily determined empirically in vitro. In particular, preferred LNA-modified oligonucleotides bind a target polynucleotide under the following moderately stringent conditions (referred to herein as “normal stringency” conditions): use of a hybridization buffer comprising 100 mM NaCl, 0.1 mM EDTA and 10 mM phosphate buffer, pH 7.0 at a temperature of 37° C. Particularly preferred LNA-modified oligonucleotides bind a target polynucleotide under the following highly stringent conditions (referred to herein as “high stringency” conditions): use of a hybridization buffer comprising 0.1 mM EDTA and 10 mM phosphate buffer, pH 7.0 at a temperature of 42° C.

Preferably, the thermal stability of hybrids formed by the LNA-modified oligonucleotides of the invention are determined by way of melting, or strand dissociation, curves. The temperature of fifty percent strand dissociation is taken as the melting temperature, Tₘ, which, in turn, provides a convenient measure of stability. Tₘ measurements are typically carried out in a saline solution at neutral pH with target and LNA-modified oligonucleotide concentrations at between about 0.5-5 μM. Typical conditions are as follows: 100 mM NaCl and 0.1 mM EDTA in a 10 mM sodium phosphate buffer (pH 7.0) and 1.5 μM of each oligonucleotide. Data for melting curves are accumulated by heating a sample of the antisense oligonucleotide/target polynucleotide complex from room temperature to about 90° C. As the temperature of the sample increases, absorbance of 260 nm light is monitored at 1° C. intervals, e.g., using a Cary (Australia) model 1E or a Hewlett-Packard (Palo Alto, Calif.) model HP 8459 UV/VIS spectrophotometer and model HP 89100A temperature controller, or like instruments. Such techniques provide a convenient means for measuring and comparing the binding strengths of LNA modified antisense oligonucleotides of different lengths and compositions.

Pharmaceutical compositions of the invention include a pharmaceutical carrier that may contain a variety of components that provide a variety of functions, including regulation of drug concentration, regulation of solubility, chemical stabilization, regulation of viscosity, absorption enhancement, regulation of pH, and the like. The pharmaceutical carrier may comprise a suitable liquid vehicle or excipient and an optional auxiliary additive or additives. The liquid vehicles and excipients are conventional and commercially available. Illustrative thereof are sterile, pyrogen-free distilled water, physiological saline, aqueous solutions of dextrose, and the like. For water soluble formulations, the pharmaceutical composition preferably includes a buffer such as a phosphate buffer, or other organic acid salt, preferably at a pH in the range of between about 6.5 to 8. For formulations containing weakly soluble antisense compounds, micro-emulsions may be employed, for example by using a nonionic surfactant such as polysorbate 80 in an amount of 0.04-0.05% (w/v), to increase solubility. Other components may include antioxidants, such as ascorbic acid, hydrophilic polymers, such as, monosaccharides, disaccharides, and other carbohydrates including cellulose or its derivatives, dextrins, chelating agents, such as EDTA, and like components well known to those in the pharmaceutical sciences, e.g., Remington's Pharmaceutical Science, latest edition (Mack Publishing Company, Easton, Pa.).

Effective therapeutics against viruses, intracellular parasites, and invasive bacteria must all be able to cross the biological membrane of the infected cell. The LNA-modified oligonucleotides would preferably penetrate the cell wall and the plasma membrane of these organisms. Penetration may be facilitated with a transporter such as a cationic peptide for example poly-L-arginine (see e.g. WO9852614 and WO9614832). These peptides may increase the LNA-modified oligonucleotides' water solubility and efficiently transport them into a wide variety of eukaryotic and prokaryotic cells with the retention of their biological activity.

LNA-modified oligonucleotides of the invention include the pharmaceutically acceptable salts thereof, including those of alkaline earth salts, e.g., sodium or magnesium, ammonium or NX₄⁺, wherein X is C₁-C₄ alkyl. Other pharmaceutically acceptable salts include organic carboxylic acids such as formic, acetic, lactic, tartaric, malic, isethionic, lactobionic, and succinic acids; organic sulfonic acids such as methanesulfonic, ethanesulfonic, toluenesulfonic acid and benzenesulfonic; and inorganic acids such as hydrochloric, sulfuric, phosphoric, and sulfamic acids. Pharmaceutically acceptable salts of a compound having a hydroxyl group include the anion of such compound with a suitable cation such as Na⁺, NH₄⁺, or the like.

LNA-modified oligonucleotides of the invention are preferably administered to a subject orally or topically but may also be administered intravenously by injection. The vehicle is designed accordingly. Alternatively, the oligonucleotide may be administered subcutaneously via controlled release dosage forms or conventional formulation for intravenous injection.

For many applications, injection or catheter administration of LNA oligonucleotide may be preferred to provide for localized administration and delivery of the therapeutic oligonucleotide.

In addition to administration with conventional carriers, an LNA oligonucleotide may be administered by a variety of specialized oligonucleotide delivery techniques. Sustained release systems suitable for use with the pharmaceutical compositions of the invention include semi-permeable polymer matrices in the form of films, microcapsules, or the like, which may comprise polylactides; copolymers of L-glutamic acid and gamma-ethyl-L-glutamate, poly(2-hydroxyethyl methacrylate), and like materials, e.g., Rosenberg et al., International application PCT/US92/05305.

The oligonucleotides may be encapsulated in liposomes for therapeutic delivery, as described for example in Liposome Technology, Vol. II, Incorporation of Drugs, Proteins, and Genetic Material, CRC Press. The oligonucleotide, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, ionic surfactants such as diacetylphosphate, stearylamine, or phosphatidic acid, and/or other materials of a hydrophobic nature. Also comprised are the novel cationic amphiphiles, termed “molecular umbrellas”, that are described in (DeLong et al, Nucl. Acid. Res., 1999, 27(16), 3334-3341).

The oligonucleotides may be conjugated to peptide carriers. Examples are poly(L-lysine) that significantly increased cell penetration and the antennapedia transport peptide. Such conjugates are described by Lemaitre et al, “Specific antiviral activity of a poly(L-lysine)-conjugated oligodeoxyribonucleotide sequence complementary to vesicular stomatitis virus N protein mRNA initiation site,” Proc. Natl. Acad. Sci. USA, 84:648-652, 1987; U.S. Pat. Nos.: 6,166,089 and 6,086,900. The procedure requires that the 3′-terminal nucleotide be a ribonucleotide. The resulting aldehyde groups are then randomly coupled to the epsilon-amino groups of lysine residues of poly(L-lysine) by Schiff base formation, and then reduced with sodium cyanoborohydride. This procedure converts the 3′-terminal ribose ring into morpholine structure antisense oligomers.

The peptide segment can also be synthesized by strategies which are compatible with DNA/RNA synthesis e.g. Mmt/Fmoc strategies. In that case the peptide can be synthesized directly before or after the oligonucleotide segment. Also methods exist to prepare the peptide oligonucleotide conjugate post synthetically, e.g., by formation of a disulfide bridge.

The LNA modified oligonucleotides may also be synthesized as pro-drugs carrying lipophilic groups, such as for example methyl-SATE (S-acetylthioethyl) or t-Bu-SATE (S-pivaloylthioethyl) protecting groups, that confers nuclease resistance to the oligo, improve cellular uptake and selectively deprotects after entry into the cell as described in Vives et al. Nucl. Acids Res. 1999, Vol. 27, 4071-4076. The LNA modified oligonucleotide may also be synthesized as circular molecules in which the 5′ and 3′ ends of the oligonucleotides are covalently linked or held together by an affinity pair one member of which is attached covalently to the 5′ end and the other attached covalently to the 3′ end. Such circularization protects the oligonucleotide against degradation by exonucleases and may also improve cellular uptake and distribution. In one aspect of the invention the moiety linking the 5′ and 3′ end of a circular oligonucleotide is cleaved automatically upon entry into any type of human or vertebrate cell thereby linearising the oligonucleotide and enabling it to efficiently hybridize to its target sequence. In another aspect, the moiety linking the 5′ and 3 ′ ends of the oligonucleotide is so designed that cleavage preferably occurs only in the particular type of cells that expresses the mRNA that is the target for the antisense oligonucleotide. For instance, a circular antisense oligonucleotide directed against a gene involved in cancer may be brought into action by linearisation only in the subset of cells expressing the malignant gene. Likewise, circular antisense oligonucleotides directed against bacterial or viral genes may be activated in only infected cells, by using for example a delivery system that targets only infected cells. Such a delivery system is described in U.S. Pat. No. 6,228,423. Other such systems have also been described Lappalainen et al., “Cationic liposomes mediated delivery of antisense oligonucleotides targeted to HPV 16 E7 mRNA in CaSki cells”, Antiviral Res., 1994, 23, 119; Mishra et al., “Improved leishmanicidal effect of phosphorotioate antisense oligonucleotides by LDL-mediated delivery”, Biochim. Biophys. Acta, 1995, 1264:229; Shea et al., “Synthesis, hybridization properties and antiviral activity of lipid-oligodeoxynucleotide conjugates”, Nucl. Acids Res., 1990, 18:3777. Other methods may include, for example, use of sequences which are specifically cleaved by endonucleases produced only in infected cells or inclusion of a covalent bond which is scission-sensitive to an intracellular enzyme activity which may be produced by a cell in response to viral infection (see for example, U.S. Pat. No. 6,166,0890) or use of an activator sequence such as an activator of RNase L (“activator-antisense complexes”) which specifically cleave a genomic or antigenomic strand of the RNA virus. See, for example, U.S. Pat. No. 6,214,805.

LNA oligonucleotides of the invention also include conjugates of such oligonucleotides with appropriate ligand-binding molecules. The oligonucleotides may be conjugated for therapeutic administration to ligand-binding molecules which recognize cell-surface molecules, such as according to International Patent Application WO 91/04753. The ligand-binding molecule may comprise, for example, an antibody against a cell surface antigen, an antibody against a cell surface receptor, a growth factor having a corresponding cell surface receptor, an antibody to such a growth factor, or an antibody which recognizes a complex of a growth factor and its receptor. Methods for conjugating ligand-binding molecules to oligonucleotides are detailed in WO 91/04753. Further, conjugation methods and methods to improve cellular uptake which may be used are described in the following international patent applications WO 9640961, WO9964449, WO9902673, WO9803533, WO0015265 and U.S. Pat. Nos. 5,856,438 and 5,138,045.

In particular, the growth factor to which the antisense oligonucleotide may be conjugated, may comprise transferrin or folate. Transferrin-polylysine-oligonucleotide complexes or folate-polylysine-oligonucleotide complexes may be prepared for uptake by cells expressing high levels of transferrin or folate receptor. The preparation of transferrin complexes as carriers of oligonucleotide uptake into cells is described by Wagner et al ., Proc. Natl. Acad. Sci. USA 87, 3410-3414 (1990). Cellular delivery of folate-macromolecule conjugates via folate receptor endocytosis, including delivery of an antisense oligonucleotide, is described by Low et al., U.S. Pat. No. 5,108,921. Also see, Leamon et al., Proc. Natl. Acad. Sci. 88, 5572 (1991).

Preferred methods of administration of oligonucleotides comprises either, topical, systemic or regional perfusion, as is appropriate. According to a method of regional perfusion, the afferent and efferent vessels supplying the extremity containing the lesion are isolated and connected to a low-flow perfusion pump in continuity with an oxygenator and a heat exchanger. The iliac vessels may be used for perfusion of the lower extremity. The axillary vessels are cannulated high in the axilla for upper extremity lesions. Oligonucleotide is added to the perfusion circuit, and the perfusion is continued for an appropriate time period, e.g., one hour. Perfusion rates of from about 100 to about 150 ml/minute may be employed for lower extremity lesions, while half that rate should be employed for upper extremity lesions. Systemic heparinization may be used throughout the perfusion, and reversed after the perfusion is complete. This isolation perfusion technique permits administration of higher doses of chemotherapeutic agent than would otherwise be tolerated upon infusion into the arterial or venous systemic circulation.

For systemic infusion, the oligonucleotides are preferably delivered via a central venous catheter, which is connected to an appropriate continuous infusion device. Indwelling catheters provide long term access to the intravenous circulation for frequent administration of drugs over extended time periods. They are generally surgically inserted into the external cephalic or internal jugular vein under general or local anesthesia. The subclavian vein is another common site of catheterization. The infuser pump may be external, or may form part of an entirely implantable central venous system such as the INFUSAPORT system available from Infusaid Corp., Norwood, Mass. and the PORT-A-CATH system available from Pharmacia Laboratories, Piscataway, N.J. These devices are implanted into a subcutaneous pocket under local anesthesia. A catheter, connected to the pump injection port, is threaded through the subclavian vein to the superior vena cava. The implant contains a supply of oligonucleotide in a reservoir which may be replenished as needed by injection of additional drug from a hypodermic needle through a self-sealing diaphragm in the reservoir. Completely implantable infusers are preferred, as they are generally well accepted by patients because of the convenience, ease of maintenance and cosmetic advantage of such devices.

LNA-modified oligonucleotides of the invention may be introduced by any of the methods described in U.S. Pat. No. 4,740,463, incorporated herein by reference. One technique is in vitro transfection, which can be done by several different methods. One method of transfection involves the addition of DEAE-dextran to increase the uptake of the naked DNA molecules by a recipient cell. See McCutchin, J. H. and Pagano, J. S., J. Natl. Cancer Inst. 41, 351-7 (1968). Another method of transfection is the calcium phosphate precipitation technique which depends upon the addition of Ca²⁺ to a phosphate-containing DNA solution. The resulting precipitate apparently includes DNA in association with calcium phosphate crystals. These crystals settle onto a cell monolayer; the resulting apposition of crystals and cell surface appears to lead to uptake of the DNA. A small proportion of the oligonucleotides taken up becomes expressed in a transfectant, as well as in its clonal descendants. See Graham, F. L. and van der Eb, A. J., Virology 52, 456-467 (1973) and Virology 54, 536-539 (1973).

Transfection of oligonucleotides may also be carried out by cationic phospholipid-mediated delivery. In particular, polycationic liposomes can be formed from N-[1-(2,3-di-oleyloxy)propyl]-N,N,N-trimethylammonium chloride (DOT-MA). See Felgner et al., Proc. Natl. Acad. Sci., 84, 7413-7417 (1987) (DNA-transfection); Malone et al., Proc. Natl. Acad Sci., 86, 6077-6081 (1989) (RNA-transfection).

A cell has been “transformed”, “transduced”, or “transfected” by exogenous or heterologous nucleic acids when such nucleic acids have been introduced inside the cell.

Particulate systems and polymers for in vitro and in vivo delivery of polynucleotides have been extensively reviewed by Felgner in Advanced Drug Delivery Reviews 5, 163-187 (1990). Techniques for direct delivery are also described in Cook S. T. Antisense Drug Technology, Principles, Strategies, and Applications, Marcel Dekker, Inc, 2001.

The LNA modified antisense oligonucleotides may be used as the primary therapeutic for the treatment of the disease state, or may be used in combination with non-oligonucleotide drugs. An antisense oligonucleotide can reduce or inhibit the expression of the genes, and thereby “reinstall” responsiveness to chemotherapeutic drugs of the otherwise resistant bacteria. Typical examples of drugs that can be used in combination with antisense oligonucleotide drugs, include drugs such as AZT, interferons and antibiotics etc.

As an illustrative example, which is not meant to limit or construe the invention in any way, the oligonucleotides of the invention are used to interfere with, for example, a retrovirus. The retroviral life cycle, as described below, offers several strategies for the interference of short oligonucleotides with virus formation and infection. The retroviral life cycle comprises the following steps:

After attachment to cellular proteins the envelope of the virus fuses with the cell membrane releasing the virion in the cytoplasm, where upon destruction of the capsid the genetic material is liberated. Retroviruses contain two plus strand RNA molecules, which are reverse transcribed in the cytoplasm by the viral enzyme reverse transcriptase. The resulting double stranded DNA molecule is integrated into the host genome by viral integrase and acts as template for synthesis of viral mRNA. The mRNA is used for protein synthesis as well as copy of the viral genome, which is finally assembled with viral proteins to form virion particles. During budding from the host cell the virion particles gain an envelope and are ready for new infection.

In the treatment of viral infections caused by retrovirus such as HIV-1, LNA-modified oligonucleotides can act in two ways, for example, as antisense oligonucleotide and as a blocking agent for viral replication.

In cells infected by retrovirus, e.g. CD4+ cells infected with HIV-1, LNA-modified antisense oligonucleotides targeting essential viral genes will downregulate the corresponding mRNAs by recruiting cellular RNase H activity and thus prevent synthesis of viral proetins, provided the LNA-modified oligo is designed to recruit such cellular RNase H activity. Additionally, binding to the viral single stranded RNA genome will recruit RNase H in the same way, resulting in destruction of the RNA and thus lack of novel virus formation. Alternatively, the high affinity of LNA-modified oligos towards their target sites in retroviral ssRNA may arrest reverse transcription of retroviral RNA into double stranded DNA, an essential step for integration of the viral genome into the host genome. The stable duplexes of retroviral RNA and LNA-modified oligos may stop the polymerisation of the minus DNA strand and thus prevent formation of the proviral DNA. Therefore, treatment of uninfected cells with LNA-modified antisense oligos targeting retroviral ssRNA will prevent these cells from infection by the virus. This mechanism is called arrest of retroviral replication.

In another illustrative non-limiting example, the oligonucleotides of the present invention can be targeted to nucleic acid molecules of various micro-organisms.

For example, infectious diseases are caused by micro-organisms belonging to a very wide range of bacteria, viruses, protozoa, worms and arthropods and LNA can be modified and used against all kinds of RNA in such micro-organisms, sensitive or resistant to antibiotics.

Examples of micro-organisms which may be treated in accordance with the present invention are Gram-positive organisms such as Streptococcus, Staphylococcus, Peptococcus, Bacillus, Listeria, Clostridium, Propionebacteria, Gram-negative bacteria such as Bacteroides, Fusobacterium, Escherichia, Klebsiella, Salmonella, Shigella, Proteus, Pseudomonas, Vibrio, Legionella, Haemophilus, Bordetella, Brucella, Campylobacter, Neisseria, Branhamella, and organisms which stain poorly or not at all with Gram's stain such as Mycobacteria, Treponema, Leptospira, Borrelia, Mycoplasma, Clamydia, Rickettsia and Coxiella, The incidence of the multiple antimicrobial resistance of bacteria which cause infections in hospitals/intensive care units is increasing. These include methicillin-resistant and methicillin-vancomycin-resistant Staphylococcus aureus, vancomycin-resistant enterococci such as Enterococcus faecalis and Enterococcus faecium, penicillin-resistant Streptococcus pneumoniae and cephalosporin and quinolone resistant gram negative rods (coliforms) such as E. coli, Klebsiella pneumoniae, Pseudomonas species and Enterobacter species. More recently, pan antibiotic (including carbapenems) resistant gram negative bacilli have emerged. The rapidity of emergence of these multiple antibiotic-resistance is not being reflected by the same rate of development of new antibiotics and it is, therefore, conceivable that patients with serious infections soon will no longer be treatable with currently available antimicrobials. Several international reports have highlighted the potential problems associated with the emergence of antimicrobial resistance in many areas of medicine and also outlined the difficulties in the management of patients with infections caused by these micro-organisms.

Gram positive bacteria Methicillin-resistant S. aureus (MRSA), methicillin-vancomycin resistant S. aureus (VMRSA) and vancomycin resistant enterococci (VRE) have emerged as major nosocomial pathogens. Vancomycin is currently the most reliable treatment for infections caused by MRSA but the potential transfer of resistance genes from VRE to MRSA may leave few therapeutic options in the future. VRE, as well as providing a reservoir of vancomycin resistance genes, can also cause infections in patients with compromised immunity, which are difficult to treat, with some strains showing resistance to all major classes of antibiotic. The increasing incidence of VRE strains among clinical isolates of enterococci places them as important nosocomial pathogens and in some hospitals in the United States VRE are responsible for more than 20% of enterococcal infections, S. aureus showing intermediate vancomycin resistance (VISA) as well as VMRSA have now been reported from several numbers of centres/hospitals worldwide.

Of the S. aureus isolates from USA, Europe and Japan 60-72% were MRSA. Most strains that are multi-drug-resistant MRSA are the most common cause of surgical site infection and comprise 61% of all such S. aureus infections and a major cause of increased morbidity and mortality of ICU patients.

Coagulase negative staphylococci (CNS) such as S. epidermidis are an important cause of infections associated with prosthetic devices and catheters. Although they display lower virulence than S. aureus, they have intrinsic low-level resistance to many antibiotics including beta-lactams and glycopeptides. In addition many of these bacteria produce slime (biofilm) making the treatment of prosthetic associated infections difficult and often requires removal of the infected prosthesis or catheter.

Streptococcus pneumoniae, regarded as fully sensitive to penicillin for many years, has now acquired the genes for resistance from oral streptococci. The prevalence of these resistant strains is increasing rapidly worldwide and this will limit the therapeutic options in serious pneumococcal infections, including meningitis and pneumonia. Streptococcus pneumoniae is the leading cause of infectious morbidity and mortality worldwide. In USA the pneumococcus is responsible for an estimated 50,000 cases of bacteremia, 3000 cases of meningitis, 7 million cases of otitis media, and several hundred thousands cases of pneumonia. The overall yearly incidence of pneumococcal bacteremia is estimated to be 15 to 35 cases per 100,000. Current immunization of small children and old people have not addressed the high incidence of pneumococcal infection. Multi-drug resistant strains were isolated in the late 1970's and are now encountered worldwide. Gram negative bacteria such as Pseudomonas aeruginosa, Pseudomonads species including Burkholderia cepacia and Xanthomonas malthophilia, Enterobacteriaceae including E. coli, Enterobacter species and Klebsiella species account for the majority of isolates where resistance has emerged. Cystitis, pneumonia, septicaemi and postoperative sepsis are the commonest types of infections. Most of the infections in patients being treated on an intensive care unit (ICU) results from the patients own endogenous flora and in addition up to 50% of ICU patients will also acquire nosocomial infection, which are associated with a relatively high degree of morbidity and mortality. Microorganisms associated with these infections include Enterobacteriaceae 34%, S. aureus 30%, P. aeruginosa 29%, CNS 19% and fungi 17%.

Selective pressure through the use of broad-spectrum antibiotics has lead to multidrug resistance in Gram-negative bacteria. Each time a new drug is introduced, resistant subclones appear and today the majority of isolates are resistant to at least one antimicrobial. The cell envelope of P. aeruginosa with the low permeability differs from that of E. coli. 46% of P. aeruginosa isolates from Europe are resistant to one or more antibiotics and the ability of this bacteria to produce slime (biofilm) and rapid development of resistance during treatment often leads to therapy failure. Multidrug resistant P. aeruginosa has also become endemic within some specialised ICU's such as those treating burns patients and cystic fibrosis patients. Several international reports have highlighted the potential problems associated with the emergence of antimicrobial resistance in bacteria mentioned above, and it is, therefore, conceivable that patients with serious infections soon will no longer be treatable with currently available antimicrobials. The increasing incidence of resistant strains among clinical isolates of S. aureus, S. epidermidis (CNS), enterococci, Streptococcus pneumoniae, gram negative bacilli (coliforms) such as E. coli, Klebsiella pneumoniae, Pseudomonas species and Enterobacter species make these bacteria major candidates for treatment with the oligonucleotides of the invention. Methods are described in the Examples which follow. (see example 5).

For systemic or regional in vivo administration, the amount of LNA-modified oligonucleotides may vary depending on the nature and extent of the disease, the particular oligonucleotides utilized, and other factors. The actual dosage administered may take into account the size and weight of the patient, whether the nature of the treatment is prophylactic or therapeutic in nature, the age, health and sex of the patient, the route of administration, whether the treatment is regional or systemic, and other factors.

The patient should receive a sufficient daily dosage of LNA modified antisense oligonucleotide to achieve an effective yet safe intercellular concentrations of combined oligonucleotides. Those skilled in the art should be readily able to derive appropriate dosages and schedules of administration to suit the specific circumstance and needs of the patient.

When a combination of LNA modified antisense oligonucleotide targeting different target sequences are employed, the ratio of the amounts of the different types of LNA modified antisense oligonucleotide may vary over a broad range. According to one preferred embodiment of the invention, the oligonucleotides of all types are present in approximately equal amounts, by molarity.

The effectiveness of the treatment may be assessed by routine methods, which are used for determining whether or not remission has occurred. Such methods generally depend upon morphological, cytochemical, cytogenetic, immunologic and molecular analyses. In addition, remission can be assessed genetically by probing the level of expression of one or more relevant genes. The reverse transcriptase polymerase chain reaction (RT-PCR) methodology can be used to detect even very low numbers of mRNA transcript. For example, RT-PCR has been used to detect and genotype HIV variants.

Typical subjects to which LNA oligonucleotides may be administered will be mammals, particularly primates, especially humans, particularly such mammals that are suffering from or susceptible to a viral, bacterial, fungal or protozoa infection or disease or disorder associated therewith. Suitably, the mammal is identified and then selected prior to administration of an LNA oligonucleotide on the basis of suffering from or susceptible to such an infection or disease or disorder.

A wide variety of subjects will be suitable for administration of an LNA oligonucleotide for veterinary applications such as e.g. livestock such as cattle, sheep, goats, cows, swine and the like; poultry such as chickens, ducks, geese, turkeys and the like; and domesticated animals particularly pets such as dogs and cats.

For diagnostic or research applications, a variety of mammals will be suitable subjects for administration of an LNA oligonucleotide in accordance with the invention, including rodents (e.g. mice, rats, hamsters), rabbits, primates, and swine such as inbred pigs and the like.

Additionally, for in vitro applications, such as in vitro diagnostic and research applications, cell samples of the of the above subjects will be suitable for use such as mammalian, particularly primate such as human, blood, urine or tissue sample, or such samples of the animals mentioned for veterinary applications. Particularly suitable will be mammalian cells of the brain, liver, kidney, heart, ovaries, testes, and the like.

The following non-limiting examples are illustrative of the invention. All documents mentioned herein are fully incorporated herein by reference.

### EXAMPLES

These following examples show the ability of LNA-modified oligonucleotides to modulate the expression of a gene from an infectious agent. The mechanisms of action of the oligonucleotides in the Examples are steric blocking or oligonucleotides that are recognised by cellular enzymes, or a combination thereof. Example 1 and 2 show the ability of LNA containing oligomers to down-regulate the expression of a specific gene in vitro. The study serves as a model for specific gene regulation of other mammalian cells and cell lines as well as viral and mycobacterial infected mammalian cells and cell lines. The chosen cell line was a transformed cell line serving as a model for studying infectious agents. Example 3 shows that LNA oligonucleotides are capable of acting as blocking agents in, for example, viral replication. Example 4 shows that a LNA-modified oligonucleotide is very potent in inhibiting expression of a Hepatitis C Virus (HCV) gene. The HCV oligonucleotide is a fully modified LNA oligonucleotide, but it can also me designed as a chimeric oligonucleotide in order to be recognizable by cellular enzymes.

### Example 1

LNA Oligonucleotide Suppresses mRNA Level in Transformed Cells Using a Transfection Vehicle

Propagating the cell line: The human cell line KU 812 was in the weeks prior to the experiment cultured in RPMI 1640, 25 mM HEPES, Glutamax-1 (cat #72400-21, Life technologies) 10% FCS, 25 μg/ml Gentamicin (Life Technologies). The cells were passaged with three days interval, and kept at a density between 3×10⁵-9×10⁵.

Transfection of the human cell line KU 812: The cell viability was determined in an Improved Neubauer cytometer by trypan blue exclusion. The cells were centrifuged at 500 g for 5 minutes in a 50 ml polypropylene tube and the medium was removed. The cell pellet was carefully resuspended in prewarmed OPTI-MEM medium (cat #51985-026, LifeTechnologies) to a density of 2.5×10⁶ cells/ml. 400 μl (1×10⁶ cells) were seeded into each well of a 24 well culture plate. 25 μM of cur 0106, (5′-G^{L}T^{L}C^{L}C^{L}A^{L}C^{L}A^{L}G^{L}C^{L}A^{L}A^{L}A^{L}C^{L}A^{L}G^{L}A^{L}-3′) a fully modified β-D ribo oxy LNA oligonucleotide, or cur 0114 (5′-gₛtₛcₛcₛaₛcₛaₛgₛcₛaₛaₛaₛcₛaₛgₛaₛ-3′) a DNA oligonucleotide with phosphorothioate backbone, were adjusted in OPTI-MEM to a concentration of 2.5 μM. Sterile H₂O was used instead of oligonucleotide for the mock transfection. Transfections were performed using lipofectin as the transfection vehicle. Lipofectin (cat #18292-037, Life Technologies) was diluted with OPTI-MEM in polystyrene tubes to solutions corresponding to 12 μg/ml final concentration in the well and left at room temperature (RT) for 45 minutes. 100 μl of the lipofectin/OPTI-MEM solution was mixed with 100 μl of each oligonucleotide, and left at RT for 15 minutes. 100 μl of the mixtures were added to the wells in duplo, and mixed by carefully pipetting. The culture plates were incubated at 37° C., in 5% CO₂ for 5 hours prior to addition of 1.5 ml RPMI 1640 (cat #72400-21, Life technologies), 25 mM HEPES, Glutamax-1, 25 μg/ml Gentamicin (Life Technologies) and 10% FCS to each well. The plates were subsequently incubated for 19 hours. The cells from each well were transferred to 2 ml microtubes and centrifuged at 7,500 rpm in a standard microcentrifuge. The supernatant was discarded and the pellet was submitted to a total RNA extraction.

RNA extraction: Total RNA was extracted using the Qiagen RNAeasy mini kit (Qiagen, Hilden Germany) according to the manufactures protocol for isolation of total RNA from animal cells. The concentration of the total RNA was determined spectrophotometrically by measuring absorption at 260 nm.

mRNA analysis by real time quantitative-PCR: First strand synthesis from 1 μg total RNAofeach sample was performed using Superscript™II RNAase H⁻ Reverse Transcriptase (cat #18064-014, Life Technologies, GibcoBRL) according to the manufactures protocol. The cDNA from the first strand synthesis was diluted 20 times, and analyzed by real time quantitative PCR in a BioRad Icycler with the sense primer 5′ aat gtc age acc aac aag tta atg a 3′(0.3 μM), antisense primer 5′ cat ccc agt tcc tcc aac ca 3′(0.3 μM) and TaqMan probe 5′FAM-aag gag cag cca gtc act gaa gac ttc ca-TAMRA 3′(0.2 μM). The primers and probe were mixed with 2×Universal TaqMan mix (cat #4304437, Applied Biosystems) and added to 3.3 μl cDNA to a final volume of 25 μl. Each sample was analyzed in triplicates. A cDNA pool reverse transcribed from 250 μg/ml total RNA from KU 812 was diluted 2 fold and used for generating a standard curve. Sterile H₂O was used instead of cDNA for the no template control. Human GAPDH gene expression was used for normalization using the human GAPDH PDAR assay (4310884E, Applied Biosystems).

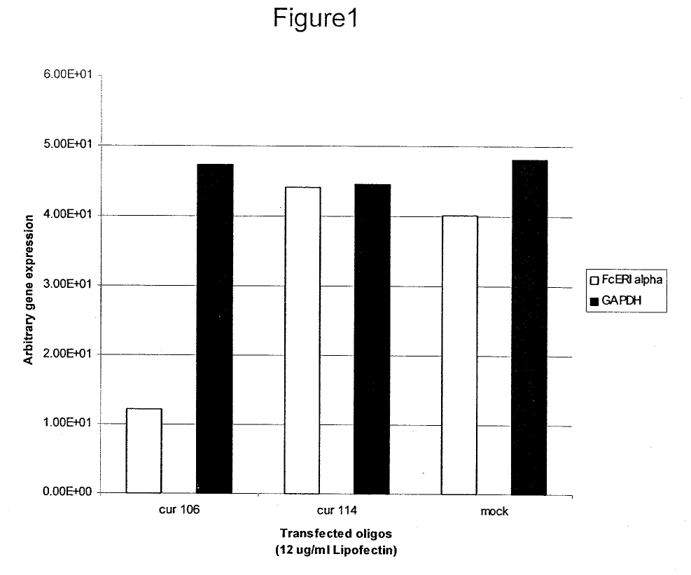

FIG. 1 shows a considerable decrease in the steady state expression using the 16-mer fully modified LNA oligonucleotide (Cur 106) compared to the 16-mer phosphorothioate oligonucleotide (cur 114) and the endogenous gene expression control (human GAPDH gene). The down-regulation of the steady state expression was approximately 3 fold, using the LNA oligonucleotide compared to the control cells (sterile H₂O without addition of oligonucleotides). Transfection of cells with the phosphorothioate cur 0114 did not result in any change in the steady state expression of this target. The transfections were made using lipofectin (12 μg/ml) as a transfection vehicle. The transfections of the oligos cur 106, cur 114 and mock (sterile H₂0) of 1×10⁶ KU 812 were performed in duplo. After 5 hours 1.5 ml RPMI 1640, (25 mM HEPES, Glutamax-1, 25 μg/ml Gentamicin) 10% FCS was added to each well. Total RNA was extracted 24 hours after transfection. The data points are plotted as mean values of three determinations in the real time PCR assay. Glyceraldehyde-3-phosphate dehydrogenase (GAPDH) was used as endogenous control.

### Example 2

LNA Oligonucleotide Suppresses mRNA Level in Transformed Cells in the Absence of a Transfection Vehicle

Propagating the cell line: The human cell line KU 812 was in the weeks prior to the experiment cultured in RPMI 1640, 25 mM HEPES, Glutamax-1 (cat #72400-21, Life technologies) 10% FCS, 25 μg/ml Gentamicin (Life Technologies). The cells were passaged with three days interval, and kept at a density between 3×10⁵-9×10⁵.

Transfection of the human cell line KU 812: The cell viability was determined in an Improved Neubauer cytometer by trypan blue exclusion. The cells were centrifuged at 500 g for 5 minutes in a 50 ml polypropylene tube and the medium was removed. The cell pellet was carefully resuspended in 37° C. OPTI-MEM medium (cat #51985-026, Life Technologies) to a density of 2.5×10⁶ cells/ml. 400 μl (1×10⁶ cells) were seeded into each well of 24 well culture plate. 25 μM solutions of cur 0106, (5′-G^{L}T^{L}C^{L}C^{L}A^{L}C^{L}A^{L}G^{L}C^{L}A^{L}A^{L}A^{L}C^{L}A^{L}G^{L}A^{L}-3′) a fully modified β-D ribo oxy LNA oligo or cur 0114 (5′-gₛtₛcₛcₛaₛcₛaₛgₛcₛaₛaₛaₛcₛaₛgₛaₛ-3′) a DNA oligonucleotide with a phosphorothioate backbone were adjusted in OPTI-MEM to a concentration of 2.5 μM. Sterile H₂O was used instead of oligonucleotide for the mock transfection. Transfections were performed without using a transfection vehicle. 100 μl of sterile H₂O was mixed with 100 μl of each oligonucleotide, and left at RT for 15 minutes. 100 μl of this mixture was added to the wells in duplo, and mixed by carefully pipetting. The culture plates were incubated at 37° C., in 5% CO₂ for 5 hours, prior to addition of 1.5 ml RPMI 1640 (cat #72400-21, Life technologies), 25 mM HEPES, Glutamax-1, 25 μg/ml Gentamicin (Life Technologies) and 10% FCS to each well. The plates were subsequently incubated for 19 hours. The cells from each well were transferred to 2 ml microtubes and centrifuged at 7,500 rpm in a standard microcentrifuge. The supernatant was discarded and the pellet was submitted to a total RNA extraction.

RNA extraction: Total RNA was extracted using the Qiagen RNeasy mini kit (Qiagen, Hilden Germany) according to the manufactures protocol for isolation of total RNA from animal cells. The concentration of the total RNA was determined spectrophotometrically by measuring absorption at 260 nm.

mRNA analysis by real time quantitative-PCR: First strand synthesis of 1 μg total RNA from each sample was performed using Superscript#II Rnase H⁻ Reverse Transcriptase (cat #18064-014, Life Technologies, GibcoBRL) according to the manufacture's protocol.

The cDNA from the first strand synthesis was diluted 20 times and analyzed by real time quantitative PCR in a BioRad Icycler with the sense primer 5′ aat gtc agc acc aac aag tta atg a 3′(0.3 μM), the antisense primer 5′ cat ccc agt tcc tcc aac ca 3′ (0.3 μM), and the Taqman probe 5′FAM-aag gag cag cca gtc act gaa gac ttc ca-TAMRA 3′(0.2 μM). The primers and probe were mixed with 2×Universal TaqMan mix (cat #4304437, Applied Biosystems) and added to 3.3 μl cDNA to a final volume of 25 μl. Each sample was prepared in triplicate. A cDNA pool reverse transcribed from 250 μg/ml total RNA from KU812 was diluted 2 fold and used for generating a standard curve. Sterile H₂O was used instead of cDNA for the no template control. Human GAPDH gene expression was used for normalization using the human GAPDH PDAR assay (4310884E, Applied Biosystems).

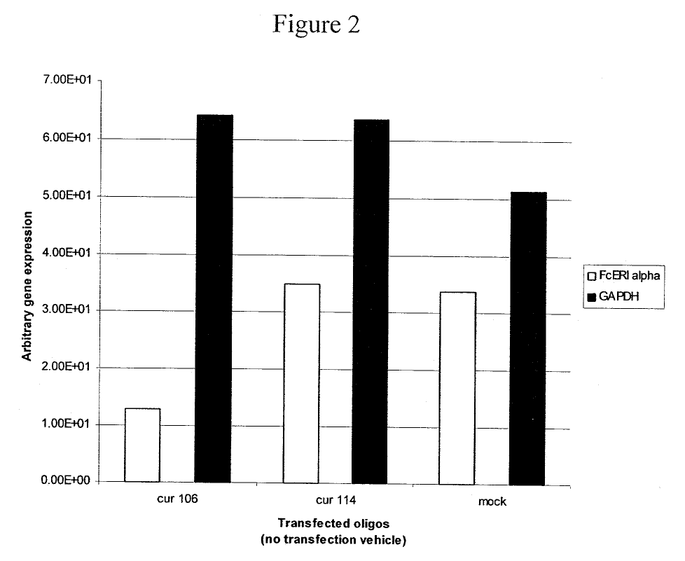

FIG. 2 shows a considerable decrease in the steady state expression using the 16-mer fully modified LNA oligonucleotide (Cur 106) compared to the 16-mer phosphorothioate oligonucleotide (cur 114) and the endogenous control (human GAPDH gene). The down-regulation in the steady state expression was approximately 3 fold, using the LNA oligonucleotide compared to the control cells (no oligonucleotides only sterile H₂O). Transfection of the phosphorothioate cur 0114, did not result in any change in the steady state expression of this target. The transfections were made without using a transfection vehicle. The transfections of the oligos cur 106, cur 114 and mock (sterile H₂O) of 1×10⁶KU 812 were performed in duplo. After 5 hours 1.5 ml RPMI 1640, (25 mM HEPES, Glutamax-1, 25 μg/ml Gentamicin) and 10% FCS was added to each well. Total RNA was extracted 24 hours after transfection. The datapoints are plotted as mean values of three determination in the real time PCR assay. GAPDH was used as an endogenous control.

In summary, the decrease in expression level was obtained both with Lipofectin as a transfection vehicle (FIG. 1) and without any transfection vehicle (FIG. 2).

### Example 3

Replicational Arrest of Retrovirus Using LNA-modified Oligonucleotides

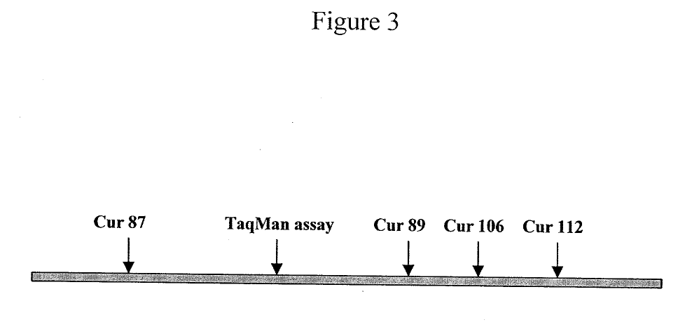

In order to test the mechanism of arrest of retroviral replication by LNA-modified oligos in vitro, RNA purified from human cells KU812 was reverse transcribed in the presence of LNA-modified oligos targeting the Fc epsilon RI alpha chain (FcεRIα) at several positions. The amount of the FcεRIα cDNA as quantified with the Taqman assay was subsequently quantified by real time PCR analysis using a FcεRFα specific Taqman assay. Of the four oligonucleotides tested one was positioned upstream and three downstream of the Taqman assay. In case of replicational arrest the amount of cDNA is expected be reduced in samples incubated with the downstream oligos and unaffected in samples incubated with the upstream oligo (see FIG. 3).

Reverse Transcription (First Strand cDNA Synthesis)

First strand cDNA synthesis was performed using Superscript™II RNase H-Reverse Transcriptase (Life Technologies, GibcoBRL). 0.5 μg total RNA was adjusted to 9 μl each with either RNase free H₂O or with Rnase free H₂O mixed with LNA antisense oligo to a final concentration of 250 nM, 83 nM, 27 nM, 9 nM, 3 nM, respectively.

To each sample 2 μl poly (dT)₁₂₋₁₈ (2.5 μg/ml) (Life Technologies, GibcoBRL) and 1 μl dNTP mix (10 mM) was added and incubated at 65° C. followed by addition of 4 μl 5×First-Strand buffer (250 mM Tris-HCl, pH 8.3 at room temp, 375 mM KCl, 15 mM MgCl2), 2 μl DTT (0.1M) and 1 μl RNAguard™RNase inhibitor (33.3 U/ml), (Amersham Pharmacia Biotech). The mixture was incubated at 42° C. for 2 minutes prior to addition of 1 μl Superscript II, (200 U/μl) followed by incubation at 42° C. for 50 minutes and heat inactivation of the enzyme at 70° C. for 15 minutes.

Real Time PCR Analysis

The cDNA from the first strand synthesis was diluted 20 times with sterile H₂O and analyzed by real time quantitative PCR. The primers and probe were mixed with 2×Universal TaqMan mix (Applied Biosystems) and added to 3.3 μl cDNA to a final volume of 25 ml. Each sample was analysed in triplicates. Standard curves were generated by assaying 2 fold dilutions of a cDNA that had been prepared from material purified from a cell line expressing the RNA of interest. Sterile H₂O was used instead of cDNA for the no template control. The following PCR program was used: 50° C. for 2 minutes, 95° C. for 10 minutes followed by 40 cycles of 95° C., 15 seconds, 60° C., 1 minute.

Relative quantities of target mRNA were determined from the calculated threshold cycle using the ABI PRISM® 7700 Sequence Detection software. The following primers and probe were used: forward primer 5′ aatgtcagcaccaacaagttaatga 3′ (final concentration 0.3 μM); reverse primer: 5′ catcccagttcctccaacca 3′ (final concentration 0.3 μM); PCR probe: 5′FAM-aaggagcagccagtcactgaagacttcca-TAMRA 3′ (final concentration 0.2 μM)

Results

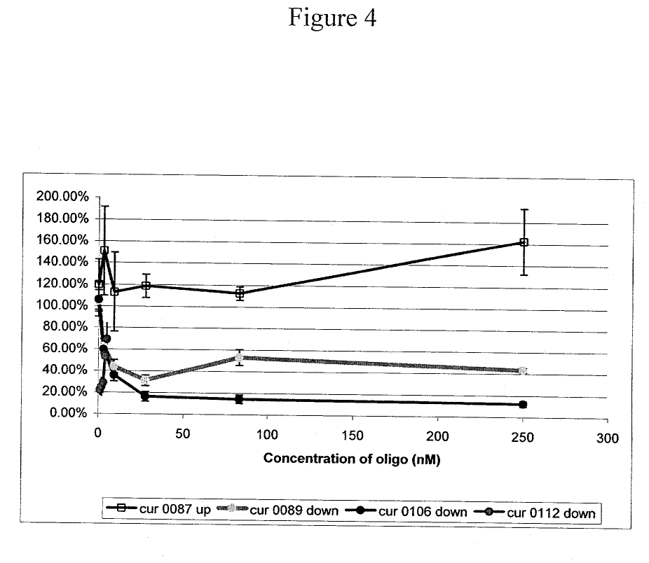

As shown in FIG. 4, a dose dependant decrease of FcεRIα cDNA was measured with the downstream oligos Cur 0089, Cur 0106 and Cur 0112, whereas the upstream oligo Cur 0087 had no effect on the FcεRIα cDNA levels. These data clearly demonstrate that the downstream oligos block reverse transcription downstream the position of the Taqman assay, whereas the upstream oligo only can block transcription upstream of the Taqman assay and thus not influence quantitation by the Taqman assay. As fully LNA-modified oligos cause replicational arrest in vitro there is a strong indication that fully LNA-modified oligos are suitable candidate drugs for the treatment of viral infections caused by retrovirus including HIV-1.

### Example 4

Inhibition Using Anti-HCVLNA Modified Antisense Oligonucleotide

An LNA modified oligonucleotide was designed to target Hepatitis C Virus (HCV) and the site was domain III of the IRES, a promotor element. This LNA oligomer is by far the most effective HCV antisense sequence compared to those previously described in the literature.

Anti-HCV LNA Modified Oligos

Cur964 is the antisense sequence and Cur0963 is the control sequence with 4 mismatches. The oligonucleotides were fully modified LNA with phosphordiester backbone. The oligos were Cur964: 5′-ACG CAA GAG TAC TCC GC-3′ and Cur963: 5′-ACC CAA CAC TAC TCG GC-3′.

Plasmids

The plasmid pgem2 HRV2 was provided by Helene Jacquemin-Sablon, Institut Bergonié, Bordeaux. It consists of the human rhinovirus 2 genomic sequence (HRV nt 10-611) followed by a coding region for a slightly truncated form of the influenza virus NS1 protein and finally the complete NS1 3′UTR (Borman and Jackson., 1992). This vector did not contain luciferase protein, so translation experiments were quantified by ³⁵S translation assay.

The dicistronic plasmid named pIRF contains the coding sequences for the firefly luciferase under the control of the cytomegalovirus promoter followed by hepatitis C Virus genomic sequence (HCV nt 1-371) and the coding sequences for the Renilla luciferase in the pcDNA3.1 Zeo vector (Invitrogen). This vector was a gift from Dr. Annie Cahour, Hopital Pitié-Salpêtrière, Paris.

In vitro Transcription

Uncapped RNA for in vitro translation assays were transcribed from the plasmids pIRF and pgem2HRV2. The pgem2HRV2 and pIRF plasmids were respectively linearised by digestion with EcoR1 and Xho1, 2 hours at 37° C., and the linearized sequences were purified by the nucleospin kit (Macherey-Nagel). After precipitation with 10 V of isopropanol, the DNA was centrifuged and pellet was dried and resuspended in 10 μl of DEPC treated water (1 μg/μl). RNAs were synthesized for 4 hours using Ampliscribe™ T7 High Yield Transcription Kit (TEBU).

IRES RNA was synthesized by in vitro transcription of DNA fragments obtained by PCR amplification from the pCV-H77 molecular clone ([Yanagi, 1997]). The polymerase chain reaction was performed with oligonucleotides primers T7 IRES and IRES3′ using 2.5 units of AmpliTaq gold DNA polymerase (Perkin Elmer) for 30 cycles. The PCR product was transcribed 4 hours at 37° C. using the MEGAscript kit (Ambion). The RNAs were precipitated and quantified by UV-absorbance at 260 nm. The RNA products were checked by electrophoresis on a polyacrylamide gel containing 7M Urea in TBE buffer (90 mM Tris-borate pH8, 1 mM EDTA).

Translation Assay

In vitro translation was performed in 30 μl of a mixture containing 15 μl of rabbit reticulocyte lysate (Promega), 2 μl of aminoacids at 1 mM and 50 ng of pIRF mRNA. This mRNA concentration was in the translation linear response region of the lysate. After 60 min incubation at 30° C. in R-buffer supplemented or not with numerous antisense oligos, the rate of translation of the renilla and firefly genes was evaluated by Dual-Luciferase Reporter Assay System (Promega) using a luminometer (Lumat Berthold).The effect of antisense was evaluated by measuring the ratio between renilla and firefly luciferases in the presence and in the absence of antisense oligos, respectively.

The pgem2HRV2 RNA (200 ng) or the pIRF RNA (300 ng) were translated with 21 μl of rabbit reticulocyte lysate (Promega), 1 μl of amino-acids without methionine, 4 μl of [³⁵S]methionine.

After 60 min incubation at 30° C. in R-buffer supplemented or not with antisense oligonucleotides, the reactions were processed for SDS polyacrylamide gel electrophoresis, and the dried gels submitted to autoradiography using Hyperfilm (Kodak).

Results

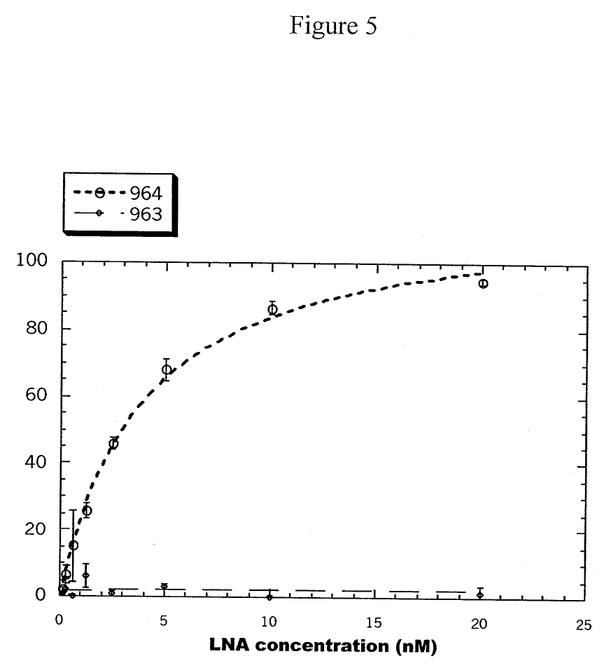

An in vitro translation cell-free assay using a bi-cistronic construct was performed. The second cistron was under the control of the HCV IRES to which the antisense oligo was targeted. The experiment has been repeated 3 times. The Cur0964 is the antisense sequence and Cur0963 is the control mismatched sequence. FIG. 5 shows the translational inhibition of the antisense oligo, compared to the control. IC50 was about 2.5 nM. This LNA oligomer is by far the most effective HCV antisense sequence compared to those previously described in the literature.

### Example 5

LNA Oligos Targeting Essential Bacterial Genes

Infectious diseases are caused by micro-organisms belonging to a very wide range of bacteria, viruses, protozoa, worms and arthropods and LNA can be modified and used against all kinds of RNA in such micro-organisms, sensitive or resistant to antibiotics. Examples of oligonucleotides useful for treatment of micro-organisms that are sensitive or resistant to antibiotics are as follows:

Full LNAs and 6s gapmers, all 12-mers with fully thiolated backbone:

Oligonucleotide design:

| | | 5′ ^{→} 3′ |
|---|---|---|
| ftsZ | | TTCAAACATAGT |
| | Cur2293 | TsTsCsAsAsAsCsAsTsAsGsT |
| infA (IF1) | | TGGCCATCTAAT |
| | Cur2207 | TsGsGsCsCsAsTsCsTsAsAsT |
| | Cur2208 | TsGsGscscsastscstsAsAsT |
| | | CTAATCCTCTGG |
| | Cur2291 | CsTsAsAsTsCsCsTsCsTsGsG |
| acpp (ACP) | | GTGCTCATACTC |
| | Cur2214 | GsTsGscstscsastsasCsTsC |

Methods

The ability of the compounds of the present invention to inhibit bacterial growth may be measured in many ways. For the purpose of exemplifying the present invention, the bacterial growth is measured by the use of a microdilution broth method according to NCCLS guidelines modified by using competent bacteria, ref: Sambrock et al., 1989. The present invention is not limited to this way of detecting inhibition of bacterial growth.

To illustrate one example of measuring growth and growth inhibition the following procedure can be used:

Bacterial strain: E. coli AS19.

Media: 100% Mueller-Hinton broth.

Trays: 96 well trays, Nunc, Copenhagen.

Competent AS19 cells are prepared using ice cold CaCl₂ and MgCl₂ according to Sambrock et al., 1989. E. coli is diluted with fresh preheated medium and adjusted to defined OD (here: Optical Density at 600 nm) in order to give a final concentration of 5×10³⁻² bacteria/ml medium in each well, containing 200 μl of bacterial culture. LNA is added to the bacterial culture in the wells in order to give final concentrations ranging from 2 μg/ml to 25.0 μg/ml. Trays are incubated at 37° C. by shaking, for 16 h. Wells containing bacterial culture without LNA are used as controls to ensure correct inoculum size and bacterial growth during the incubation. Cultures are tested in order to detect contamination.

Growth Inhibitory Effect of LNA-constructs:

Total inhibition of bacterial growth is defined as no visible growth seen by the naked eye according to NCCLS Guidelines.

Minimal Inhibitory Concentration and Minimal Bactericidal Concentration: In addition experiments were carried out to evaluate the relationship between MIC's and MBC's (Minimal Bactericidal Concentration) of the LNA.

Experimental Setup for MBC:

MIC's was detected as previously described. Trays were incubated at 35 0 C. for further 24 h in order to analyze regrowth of inhibited bacteria (MBC's).

MIC and MBC Assay with LNA 2203, 2207, 2208, 2214 and 2291:

The assay was performed as previously described.

Results:

Total inhibition of growth using the above-mentioned LNA oligonucleotides can be seen in cultures with 10³ bacterial cells/ml and a LNA concentration of at least 20 ug/ml.

MIC values are equal to MBC values.

### Example 6

LNA Oligonucleotides and Virus Assay:

Viruses and Cells.

The HSV-1 strain McIntyre was propagated in VERO cells at 37° C., 5% CO₂ using RPMI 1640 with heat-inactivated fetal calf serum (FCS) and antibiotics (growth medium). Culture supernatant was filtered (0.45 nm), aliquotted, and stored at −80° C. until use. The HSV-1 strain and VERO cells were obtained from ATCC.

Inhibition of HIV-1 Replication.

Compounds were examined for possible antiviral activity against the HSV-1 strain using VERO cells as target cells. VERO cells were incubated with growth medium containing the test dilutions of compound for one hour. Subsequently a standardized titer of virus was added and the cultures were incubated for six days, at 37° C., 5% CO₂ in parallel with virus-infected and uninfected control cultures without compound added. Expression of HSV-1 in the cultures was indirectly quantified using the MTT assay as previously described. Compounds mediating less than 30% reduction of HIV expression were considered without biological activity. Compounds were tested in parallel for cytotoxic effect in uninfected VERO cell cultures containing the test dilutions of compound as described above. A 30% inhibition of cell growth relative to control cultures was considered significant. The 50% inhibitory concentration (IC50) and the 50% cytotoxic concentration (CC50) were determined by interpolation from the plots of percent inhibition versus concentration of compound.

The invention has been described in detail with reference to preferred embodiments thereof. However, it will be appreciated that those skilled in the art, upon consideration of this disclosure, may make modifications and improvements within the spirit and scope of the invention.

What is claimed is:

1. A method of inhibiting propagation of an infectious agent associated with an infectious disease, comprising;
contacting the infectious agent with an oligonucleotide that comprises one or more LNA units;
wherein contacting of the LNA oligonucleotide to the infectious agent modulates expression of a gene of the infectious agent.

2. The method of claim 1 wherein the LNA oligonucleotide inhibits expression of a gene of the infectious agent.

3. The method of claim 1 or 2 wherein contacting of the LNA oligonucleotide inhibits the functionality of a gene involved in the pathogenesis of the infectious agent

4. The method of any one of claims 1 through 3 wherein the LNA oligonucleotide is complementary to a gene of the infectious agent.

5. A method of modulating expression of a gene from an infectious agent associated with an infectious disease, comprising;
contacting an oligonucleotide sequence of an infectious agent with a complementary oligonucleotide sequence that comprises one or more LNA units;
whereby contacting of the LNA oligonucleotide sequence to the infectious agent modulates expression of a gene coding for the infectious agent.

6. The method of any one of claims 1 through 5 wherein the contacting inhibits infectious agent protein or peptide production associated with propagation of the infectious agent.

7. The method of any one of claims 1 through 6 wherein the LNA oligonucleotide is administered to cells or tissue that comprises the infectious agent.

8. The method of any one of claims 1 through 7 wherein the infectious agent gene or oligonucleotide comprises single stranded DNA, double stranded DNA, cDNA or RNA.

9. The method of any one of claims 1 through 8 wherein the infectious agent is a virus.

10. The method of any one of claims 1 to 9 wherein contacting with the LNA oligonucleotide inhibits viral protein synthesis, viral cell membrane synthesis, viral nucleic acid synthesis, viral replication, or viral genes encoding host immune modulating functions.

11. The method of any one of claims 1 to 10 wherein the infectious agent gene comprises at least a portion of a sequence identified in table 3 above.

12. The method of any one of claims 1 through 8 wherein the infectious agent is a bacterium.

13. The method of claim 12 wherein the bacterium is identified in table 4 above.

14. The method of claim 12 or 13 wherein the bacterium comprises a gene or oligonucleotide having a sequence that at least a portion of which is identified in table 5 or 6 above.

15. The method of any one of claims 1 through 8 wherein the infectious agent is or associated with a protozoa or fungi.

16. The method of any one of claims 1 through 15 wherein the LNA oligonucleotide hybridizes with messenger RNA of a gene or oligonucleotide of the infectious agent to inhibit expression thereof.

17. The method of any one of claims 1 through 16 wherein the LNA oligonucleotide is administered to mammalian cells.

18. A method of treating a mammal suffering from or susceptible to an infectious disease or disorder, comprising:
administering to the mammal a therapeutically effective amount of an oligonucleotide that comprises one or more LNA units.

19. The method of claim 19 wherein the infectious disease is caused by or associated with a virus, bacteria, protozoa or fungi.

20. The method of claim 19 or 20 wherein the infectious agent is present in a lung, heart, liver, stomach, intestine, bowel, prostate, brain, spinal cord, sinuses, urinary tract or ovaries of the mammal.

21. The method of any one of claims 18 through 20 wherein the disease or disorder is associated with undesired expression of at least a portion of a sequence identified in table 2, 4 or 5 above.

22. The method of any one of claims 18 through 21 wherein the administered LNA oligonucleotide hybridizes with messenger RNA of the gene to inhibit expression thereof.

23. The method of any one of claims 18 through 22 wherein administering the LNA oligonucleotide results in inhibition of gene expression.

24. The method of any one of claims 1 through 23 wherein the LNA oligonucleotide contains a total of from about 8 to about 100 base units.

25. The method of any one of claims 1 through 23 wherein the LNA oligonucleotide contains a total of from about 8 to about 60 base units.

26. The method of any one of claims 1 through 23 wherein the LNA oligonucleotide contains a total of from about 10 to about 40 base units.

27. The method of any one of claims 1 through 23 wherein the LNA oligonucleotide contains a total of from about 10 to about 20 base units.

28. The method of any one of claims 1 through 27 wherein the LNA oligonucleotide contains a total of 1, 2, 3, 4, 5, 6, 7, 8, 9 or 10 LNA units.

29. The method of any one of claims 1 through 28 wherein the LNA oligonucleotide comprises one or more units of Formula I as that Formula I is defined above.

30. The method of any one of claims 1 through 29 wherein the LNA oligonucleotides comprises one or more units of scheme II as that scheme II is defined above.

31. A method for treating cells comprising an infectious agent, comprising:
administering to the cells an oligonucleotide sequence that comprises one or more LNA units, the cells comprising an oligonucleotide sequence of an infectious agent.

32. The method of claim 31 wherein the LNA oligonucleotide sequence is complementary to the infectious agent oligonucleotide sequence.

33. The method of claim 31 or 32 wherein the cells are mammalian cells.

34. The method of any one of claims 31 through 33 wherein the cells are infected with a bacteria, protozoa or fungi.

35. The method of any one of claims 31 through 34 wherein the LNA oligonucleotide contains a total of from about 8 to about 100 base units.

36. The method of any one of claims 31 through 34 wherein the LNA oligonucleotide contains a total of from about 8 to about 60 base units.

37. The method of any one of claims 31 through 34 wherein the LNA oligonucleotide contains a total of from about 10 to about 40 base units.

38. The method of any one of claims 31 through 34 wherein the LNA oligonucleotide contains a total of from about 10 to about 20 base units.

39. The method of any one of claims 31 through 38 wherein the LNA oligonucleotide contains a total of 1, 2, 3, 4, 5, 6, 7, 8, 9 or 10 LNA units.

40. The method of any one of claims 31 through 39 wherein the LNA oligonucleotide comprises one or more units of Formula I as that Formula I is defined above.

41. The method of any one of claims 31 through 40 wherein the LNA oligonucleotides comprises one or more units of scheme II as that scheme II is defined above.

42. Use of an LNA oligonucleotide for the preparation of a medicament for the preparation of an agent for the treatment of a bacterial, protozoa or fungi infection.

43. The use of claim 42 wherein the LNA oligonucleotide contains a total of from about 8 to about 100 base units.

44. The use of claim 42 wherein the LNA oligonucleotide contains a total of from about 8 to about 60 base units.

45. The use of claim 42 wherein the LNA oligonucleotide contains a total of from about 8 to about 40 base units.

46. The use of claim 42 wherein the LNA oligonucleotide comprises from about 10 to about 20 base units.

47. The use of any one of claims 42 through 46 wherein the LNA oligonucleotide contains a total of 1, 2, 3, 4, 5, 6, 7, 8, 9 or 10 LNA units.

48. The use of any one of claims 42 through 47 wherein the LNA oligonucleotide comprises one or more units of Formula I as that Formula I is defined above.

49. The use of any one of claims 42 through 48 wherein the LNA oligonucleotide comprises one or more units of scheme II as that scheme is defined above.

50. A pharmaceutical composition comprising an LNA oligonucleotide packaged together with written instructions for use of the oligonucleotide for the treatment of a disease or disorder associated with a bacterial, protozoa or fungi infection.

51. The pharmaceutical composition of claim 50 wherein the LNA oligonucleotide contains a total of from about 8 to about 100 base units.

52. The pharmaceutical composition of claim 50 wherein the LNA oligonucleotide contains a total of from about 8 to about 60 base units.

53. The pharmaceutical composition of claim 50 wherein the LNA oligonucleotide contains a total of from about 10 to about 40 base units.

54. The pharmaceutical composition of claim 50 wherein the LNA oligonucleotide comprises from about 10 to about 20 base units.

55. The pharmaceutical composition of any one of claims 50 through 54 wherein the LNA oligonucleotide contains a total of 1, 2, 3, 4, 5, 6, 7, 8, 9 or 10 LNA units.

56. The pharmaceutical composition of any one of claims 50 through 55 wherein the LNA oligonucleotide comprises one or more units of Formula I as that Formula I is defined above.

57. The pharmaceutical composition of any one of claims 50 through 56 wherein the LNA oligonucleotide comprises one or more units of scheme II as that scheme is defined above.
